(19) United States
(12) Patent Application Publication
(10) Pub. No.: US 2021/0087175 A1
(43) Pub. Date: Mar. 25, 2021

(54) Crystalline Form II of Darolutamide

(71) Applicant: Sandoz AG, Basel (CH)

(72) Inventors: Verena Adamer, Kundl (AT); Nolwenn Martin, Kundl (AT); Andrea Thaler, Kundl (AT); Veronika Werner, Kundl (AT); Johannes Raneburger, Kundl (AT); Franz Xaver Schwarz, Kundl (AT); Igor Legen, Ljubljana (SI); Rebeka Jereb, Ljubljana (SI)

(73) Assignee: Sandoz AG, Basel (CH)

(21) Appl. No.: 16/971,908

(22) PCT Filed: Feb. 25, 2019
(86) PCT No.: PCT/EP2019/054638

§ 371 Date: Aug. 21, 2020

(30) Foreign Application Priority Data
Feb. 27, 2018 (EP) 18158915.1
Apr. 25, 2018 (EP) 18169249.2

(51) Int. Cl. C07D 403/12

(52) U.S. Cl. CPC C07D 403/12; A61K 9/0053; C07B 2200/13

(57) ABSTRACT

The present invention relates to a crystalline form of darolutamide and to a process for its preparation. Furthermore, the invention relates to a pharmaceutical composition comprising said crystalline form of darolutamide, preferably in a predetermined and/or effective amount, and at least one pharmaceutically acceptable excipient. The present invention also relates to 5 pharmaceutical compositions comprising darolutamide at a high drug load. The pharmaceutical composition of the present invention can be used as a medicament, in particular for the treatment of prostate cancer.

### FIELD OF THE INVENTION

The present invention relates to a crystalline form of darolutamide and to a process for its preparation. Furthermore, the invention relates to a pharmaceutical composition comprising said crystalline form of darolutamide, preferably in a predetermined and/or effective amount, and at least one pharmaceutically acceptable excipient. The present invention also relates to pharmaceutical compositions comprising darolutamide at a high drug load. The pharmaceutical composition of the present invention can be used as a medicament, in particular for the treatment of prostate cancer.

### BACKGROUND OF THE INVENTION

Prostate cancer is the most common cancer and among the three leading causes of cancer deaths in men in the United States and in Europe. The activation of androgen receptor signaling is crucial for prostate cancer growth at all states of the disease [Moilanen, A.-M. et al. Discovery of ODM-201, a new-generation androgen receptor inhibitor targeting resistance mechanisms to androgen signaling-directed prostate cancer therapies. Sci. Rep. 5, 12007; doi: 10.1038/srep12007 (2015)].

Daroutamide, chemically also known as N-{(2S)-1-[3-(3-chloro-4-cyanophenyl)-1H-pyrazol-1-yl]-propan-2-yl}-5-[(1RS)-1-hydroxyethyl]-1H-pyrazole-3-carboxamide, is a second generation orally active nonsteroidal androgen receptor antagonist currently tested in phase 3 clinical trials against nonmetastatic castration-resistant prostate cancer (CRPC). It can be represented by the following chemical structure as depicted in Formula (1)

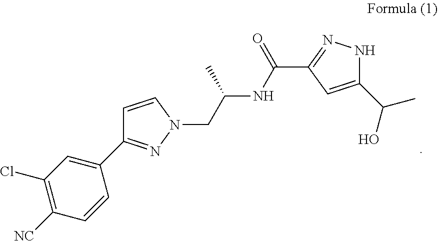 [SMILES: CC(O)c1cc(C(=O)N[C@@H](C)Cn2ccc(-c3ccc(C#N)c(Cl)c3)n2)n[nH]1]

Darolutamide comprises a mixture of two pharmacologically active diastereomers represented by the chemical structure as depicted in Formula (1a)

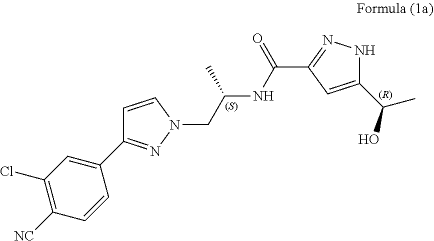

and the chemical structure as depicted in Formula (1b)

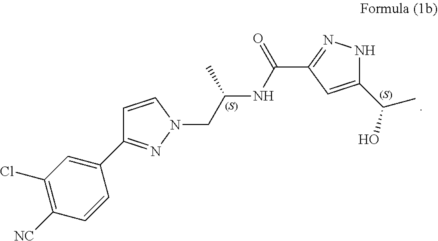

The two diastereomers interconvert via the primary pharmacologically active metabolite of darolutamide represented by the chemical structure as depicted in Formula (2)

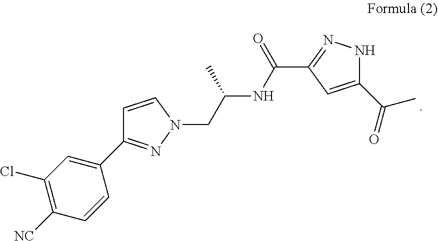 [SMILES: CC(=O)c1cc(C(=O)N[C@@H](C)Cn2ccc(-c3ccc(C#N)c(Cl)c3)n2)n[nH]1]

Darolutamide and its preparation are disclosed in WO 2011/051540 A1. A crystalline Form I of darolutamide as well as a crystalline Form I′ and a crystalline Form I″ of its diastereomers and methods for their preparation are disclosed in WO 2016/120530 A1. CN107602471A discloses an alternative process for the preparation of crystalline Form I of darolutamide.

Different solid state forms of an active pharmaceutical ingredient often possess different properties. Differences in the physicochemical properties of solid state forms can be important for the improvement of pharmaceutical compositions, for example, pharmaceutical formulations with improved dissolution profile or with improved stability or shelf-life can become accessible due to an improved solid state form of an active pharmaceutical ingredient. Also processing or handling of the active pharmaceutical ingredient during the formulation process may be improved. New solid state forms of an active pharmaceutical ingredient can thus have desirable processing properties. They can be easier to handle, better suited for storage, and/or allow for better purification, compared to previously known solid state forms.

Crystalline Form I of darolutamide of WO 2016/120530 A1 was found to possess low crystallinity and significant amorphous content, which is critical with regards to solid form transformations during pharmaceutical processing and or storage. Such material is also often hygroscopic and electrostatically charged and therefore difficult to handle. In addition, Form I consists of heterogenous particles with undefined morphology, which translates to poor powder properties, in particular poor flowability and compactibility. Hence, formulating an oral solid dosage form comprising Form I is challenging, especially when using standard formulation techniques and equipment. Furthermore, the crystalline Form I of darolutamide of WO 2016/120530 A1 is a modification of low solubility, a fact which is especially critical for the oral bioavailability of a low soluble compound like darolutamide.

It is thus an objective of the present invention to provide an improved polymorph of darolutamide, in particular a polymorph of darolutamide having improved powder properties such as crystallinity, polymorphic purity, hygroscopicity, flowability and compactibility, which enhances the processability of darolutamide during drug product manufacture. Another objective of the present invention is to provide an improved polymorph of darolutamide, e.g. a polymorph with increased solubility, which enhances the bioavailability of darolutamide.

### SUMMARY OF THE INVENTION

The present invention solves one or more of the above mentioned problems by providing a polymorph of darolutamide, which is hereinafter also referred to “Form II”. Form II of the present invention is obtained in pure polymorphic form, possesses high crystallinity and consists of well-defined crystals with homogenous morphology, which translates to excellent powder properties for the manufacture of an oral solid dosage form. In addition, darolutamide Form II of the present invention possesses increased solubility in physiologically relevant aqueous media compared to Form I of WO 2016/120530 A1, which is a crucial advantage for a drug substance having low solubility like darolutamide in order to reach the desired in vivo blood levels. The fact that darolutamide Form II of the present invention combines excellent powder properties with increased solubility renders this form a preferred modification of darolutamide for the manufacture of a safe and efficacious drug product comprising darolutamide.

### Abbreviations

PXRD powder X-ray diffractogram
FTIR Fourier transform infrared
ATR attenuated total reflection
DSC differential scanning calorimetry
TGA thermogravimetric analysis
DMF dimethylformamide
GMS gravimetric moisture sorption

### Definitions

The term “darolutamide” as used herein refers to N-{(2S)-1-[3-(3-chloro-4-cyanophenyl)-1H-pyrazol-1-yl]-propan-2-yl}-5-[(1RS)-1-hydroxyethyl]-1H-pyrazole-3-carboxamide according to Formula (1) disclosed herein above. The term “darolutamide” as used herein emcompasses both entantiomers, namely N-{(2S)-1-[3-(3-chloro-4-cyanophenyl)-1H-pyrazol-1-yl]-propan-2-yl}-5-[(1R)-1-hydroxyethyl]-1H-pyrazole-3-carboxamide according to Formula (1a) and N-{(2S)-1-[3-(3-chloro-4-cyanophenyl)-1H-pyrazol-1-yl]-propan-2-yl}-5-[(1S)-1-hydroxyethyl]-1H-pyrazole-3-carboxamide according to Formula (1b) disclosed herein above. The term “darolutamide” as used herein also includes tautomeric forms of darolutamide.

The terms “crystalline Form I of darolutamide” or “Form I of darolutamide” or “Form I” as used interchangeably herein, refer to the crystalline form of darolutamide, which is disclosed and also designated as Form I in WO 2016/120530 A1. Form I of darolutamide can be characterized by having a powder X-ray diffractogram comprising reflections at 2-Theta angles of (8.5±0.2)°, (10.4±0.2)°, (16.6±0.2)°, (16.9±0.2)° and (24.3±0.2)°, when measured at a temperature in the range of from 20 to 30° C. with Cu-Kalpha_{1,2} radiation having a wavelength of 0.15419 nm.

As used herein, the term “measured at a temperature in the range of from 20 to 30° C.” refers to a measurement under standard conditions. Typically, standard conditions mean a temperature in the range of from 20 to 30° C., i.e. at room temperature. Standard conditions can mean a temperature of about 22° C.

The term “reflection” with regard to powder X-ray diffraction as used herein, means peaks in an X-ray diffractogram, which are caused at certain diffraction angles (Bragg angles) by constructive interference from X-rays scattered by parallel planes of atoms in solid material, which are distributed in an ordered and repetitive pattern in a long-range positional order. Such a solid material is classified as crystalline material, whereas amorphous material is defined as solid material, which lacks long-range order and only displays short-range order, thus resulting in broad scattering. According to literature, long-range order e.g. extends over approximately 100 to 1000 atoms, whereas short-range order is over a few atoms only (see “Fundamentals of Powder Diffraction and Structural Characterization of Materials” by Vitalij K. Pecharsky and Peter Y. Zavalij, Kluwer Academic Publishers, 2003, page 3).

As used herein, the term “amorphous” refers to a solid form of a compound that is not crystalline. An amorphous compound possesses no long-range order and does not display a definitive X-ray diffraction pattern with reflections.

The term “essentially the same” with reference to powder X-ray diffraction means that variabilities in reflection positions and relative intensities of the reflections are to be taken into account. For example, a typical precision of the 2-Theta values is in the range of 0.2° 2-Theta, preferably in the range of 0.1° 2-Theta. Thus, a reflection that usually appears at 16.4° 2-Theta for example can appear between 16.2° and 16.6° 2-Theta, preferably between 16.3 and 16.5° 2-Theta on most X-ray diffractometers under standard conditions. Furthermore, one skilled in the art will appreciate that relative reflection intensities will show inter-apparatus variability as well as variability due to degree of crystallinity, preferred orientation, sample preparation and other factors known to those skilled in the art and should be taken as qualitative measure only.

The term “essentially the same” with reference to Fourier transform infrared spectroscopy means that variabilities in peak positions and relative intensities of the peaks are to be taken into account. For example, a typical precision of the wavenumber values is in the range of 2 cm⁻¹. Thus, a peak at 2233 cm⁻¹ for example can appear in the range of from 2231 to 2235 cm⁻¹ on most infrared spectrometers under standard conditions. Differences in relative intensities are typically smaller compared to X-ray diffraction. However, one skilled in the art will appreciate that small differences in peak intensities due to degree of crystallinity, sample preparation and other factors can also occur in infrared spectroscopy. Relative peak intensities should therefore be taken as qualitative measure only.

As used herein, the term “substantially free of any other physical form” with reference to a composition comprising a particular physical form of darolutamide means that the composition includes at most 20%, preferably at most 10%, more preferably at most 5%, even more preferably at most 2% and most preferably at most 1% by weight of any other physical form of darolutamide, based on the weight of the composition.

The term “physical form” as used herein refers to any crystalline and/or amorphous phase of a compound.

The terms “anhydrous form” or “anhydrate” as used herein refer to a crystalline solid where no water is cooperated in or accommodated by the crystal structure. Anhydrous forms may still contain residual water, which is not part of the crystal structure but may be adsorbed on the surface or absorbed in disordered regions of the crystal. Typically, an anhydrous form does not contain more than 2.0 weight %, preferably not more than 1.0 weight % and most preferably not more than 0.5 weight % of water, based on the weight of the crystalline form. The water content can be determined by Karl-Fischer Coulometry.

The term “non-solvated” as used herein, when talking about a crystalline solid indicates that no organic solvent is cooperated in or accommodated by the crystal structure. Non-solvated forms may still contain residual organic solvents, which are not part of the crystal structure but may be adsorbed on the surface or absorbed in disordered regions of the crystal. Typically, a non-solvated form does not contain more than 2.0 weight %, preferably not more than 1.0 weight % and most preferably not more than 0.5 weight % of organic solvents, based on the weight of the crystalline form. The organic solvent content can be determined by thermogravimetric analysis (TGA), e.g. by determining the weight loss in the range of from 25° C. to the melting point of the solid form at a heating rate of 10 K/min and/or by gas chromatography.

Crystalline forms of darolutamide may be referred to herein as being characterized by a powder X-ray diffractogram or a FTIR spectrum “as shown in” a figure. The person skilled in the art understands that factors such as variations in instrument type, response and variations in sample directionality, sample concentration, sample purity, sample history and sample preparation may lead to variations, for example relating to the exact reflection and peak positions and their intensities. However, a comparison of the graphical data in the figures herein with the graphical data generated for an unknown physical form and the confirmation that two sets of graphical data relate to the same crystal form is well within the knowledge of a person skilled in the art. As used herein, the term “mother liquor” refers to the solution remaining after crystallization of a solid from said solution.

The term “antisolvent” as used herein refers to a liquid, which reduces the solubility of darolutamide in a solvent.

The terms “lath” or “lath-shaped” as used herein with regard to crystal morphology refer to elongated, thin and blade-like crystals.

The terms “needle” or “needle-shaped” as used herein with regard to crystal morphology refer to acicular, thin and highly elongated crystals having similar width and breadth.

The term “spherulitic” as used herein with regard to particle shape refers to a sphere shaped particle with needles or laths radiating from the centre.

A “predetermined amount” as used herein with regard to darolutamide refers to the initial amount of darolutamide used for the preparation of a pharmaceutical composition having a desired dosage strength of darolutamide.

The term “effective amount” as used herein with regard to darolutamide encompasses an amount of darolutamide, which causes the desired therapeutic and/or prophylactic effect.

As used herein, the term “about” means within a statistically meaningful range of a value. Such a range can be within an order of magnitude, typically within 10%, more typically within 5%, even more typically within 1% and most typically within 0.1% of the indicated value or range. Sometimes, such a range can lie within the experimental error, typical of standard methods used for the measurement and/or determination of a given value or range.

The term “pharmaceutically acceptable excipient” as used herein refers to substances, which do not show a significant pharmacological activity at the given dose and that are added to a pharmaceutical composition in addition to the active pharmaceutical ingredient.

For the purpose of this invention, particle size distribution is determined as the percent volume at each particle size and measured by a laser diffraction method using a Malvern Mastersizer 3000 laser diffraction analyzer.

d90 as used herein means that 90% of the particles (based on volume) are smaller than or equal to the indicated size. d50 as used herein means that 50% of the particles (based on volume) are smaller than or equal to the indicated size. d10 as used herein means that 10% of the particles (based on volume) are smaller than or equal to the indicated size.

### BRIEF DESCRIPTION OF THE FIGURES

FIG. 1: illustrates a representative PXRD of crystalline Form II of darolutamide of the present invention. The x-axis shows the scattering angle in °2-Theta, the y-axis shows the intensity of the scattered X-ray beam in counts of detected photons.

FIG. 2: illustrates a representative FTIR spectrum of crystalline Form II of darolutamide of the present invention. The x-axis shows the wavenumbers in cm⁻¹, the y-axis shows the relative intensity in percent transmittance.

FIG. 3: illustrates a representative DSC curve of crystalline Form II of darolutamide of the present invention. The x-axis shows the temperature in degree Celsius (° C.), the y-axis shows the heat flow rate in Watt per gram (W/g) with endothermic peaks going up.

FIG. 4: illustrates a representative TGA curve of crystalline Form II of darolutamide of the present invention. The x-axis shows the temperature in degree Celsius (° C.), the y-axis shows the mass of the sample in weight percent (weight %).

FIG. 5a: illustrates a scanning electron microscopic image of crystalline Form II of darolutamide of the present invention (magnification: ×400; scale bar: 200 micrometer).

FIG. 5b: illustrates a scanning electron microscopic image of crystalline Form II of darolutamide of the present invention (magnification: ×800; scale bar: 100 micrometer).

FIG. 6: illustrates a comparison of a representative PXRD of crystalline Form II of darolutamide of the present invention (bottom) and a representative PXRD of crystalline Form I of darolutamide of WO 2016/120530 A1 (top). The x-axis shows the scattering angle in °2-Theta, the y-axis shows the intensity of the scattered X-ray beam in counts of detected photons. The PXRD of Form I was shifted along the y-axis to separate the diffractograms for clarity.

FIG. 7: illustrates a representative DSC curve of crystalline Form I of darolutamide of WO 2016/120530 A1. The x-axis shows the temperature in degree Celsius (° C.), the y-axis shows the heat flow rate in Watt per gram (W/g) with endothermic peaks going up.

FIG. 8: illustrates a representative TGA curve of crystalline Form I of darolutamide of WO 2016/120530 A1. The x-axis shows the temperature in degree Celsius (° C.), the y-axis shows the mass of the sample in weight percent (weight %).

FIG. 9a: illustrates a scanning electron microscopic image of crystalline Form I of darolutamide of WO 2016/120530 A1 (magnification: ×400; scale bar: 200 micrometer).

FIG. 9b: illustrates a scanning electron microscopic image of crystalline Form I of darolutamide of WO 2016/120530 A1 (magnification: ×800; scale bar: 100 micrometer).

FIG. 10: Time-dependent solubility curve of darolutamide form I and form II in aqueous hydrochloric acid (0.1 N) at 37° C. The x-axis shows the time in minutes (min), the y-axis shows the concentration of darolutamide in solution in micrograms per milliliter (μg/mL).

FIG. 11: Time-dependent solubility curve of darolutamide form I and form II in phosphate buffer (pH 7.5) at 37° C. The x-axis shows the time in minutes (min), the y-axis shows the concentration of darolutamide in solution in micrograms per milliliter (μg/mL).

FIG. 12: Calibration curve for solubility determination by HPLC of darolutamide. The x-axis shows the concentration of darolutamide in the prepared solutions in micrograms per milliliter (μg/mL), the y-axis shows the value of the peak area determined by HPLC in arbitrary units (a.u.).

FIG. 13: GMS equilibrium curve of darolutamide form I. The x-axis shows the relative humidity in percent (%), the y-axis shows the relative water content of darolutamide form I in percent (%).

FIG. 14: GMS equilibrium curve of darolutamide form II. The x-axis shows the relative humidity in percent (%), the y-axis shows the relative water content of darolutamide form II in percent (%).

FIG. 15: Dissolution profile of darolutamide form I tablets (300 mg) with different particle sizes of API (fine, coarse and not sieved; USP II, FeSSIF, pH 5.0, 900 mL, 37° C., 75 rpm). The x-axis shows the time in minutes, the y-axis shows the drug release of darolutamide form I in percent (%) of darolutamide form I's maximal dissolution

FIG. 16: Dissolution profile of darolutamide form I tablets (600 mg) with different particle sizes of API (fine and not sieved) and different drug load (USP II, Phosphate buffer pH 7.0+2.0 wt % SDS, 900 mL, 37° C., 75 rpm). The x-axis shows the time in minutes, the y-axis shows the drug release of darolutamide form I in percent (%) of darolutamide form I's maximal dissolution.

FIG. 17: Dissolution profile of darolutamide form II tablets (300 mg and 600 mg) with crystalline darolutamide form II and different drug load (USP II, FeSSIF, pH 5.0, 900 mL, 37° C., 75 rpm). The x-axis shows the time in minutes, the y-axis shows the drug release of darolutamide in milligram (mg).

### DETAILED DESCRIPTION OF THE INVENTION

The present invention provides a polymorph of darolutamide, herein also designated as “Form II”.

Form II of darolutamide of the present invention possesses desirable physicochemical properties for a drug substance intended for use in an oral solid dosage form. Said properties include chemical stability, physical stability, hygroscopicity, solubility, dissolution, morphology, crystallinity, flowability, compactibility and wettability. In particular, Form II of the present invention is a phase pure highly crystalline form of darolutamide with excellent physicochemical stability during pharmaceutical processing and storage. Form II consists of crystals with well-defined morphology, said morphology leading to excellent powder properties and processability allowing the formulation of a drug product comprising Form II via standard manufacturing processes and equipment. In addition, Form II of the present invention shows higher solubility compared to Form I of WO 2016/120530 A1, which may translate to higher bioavailability, which is a crucial advantage for a low soluble drug substance like darolutamide. Hence, Form II of the present invention combines high physicochemical stability with excellent powder properties and high solubility and is therefore an excellent solid form of darolutamide for the standard manufacturing of a pharmaceutical composition comprising darolutamide, even allowing the preparation of an improved pharmaceutical composition e.g. a pharmaceutical composition comprising darolutamide with increased bioavailability.

Darolutamide can be represented by the following chemical structure according to Formula (1)

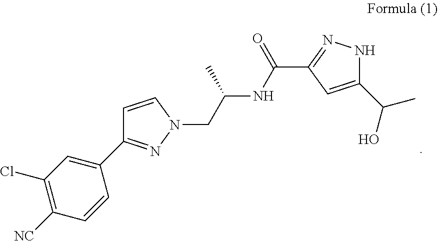 [SMILES: CC(O)c1cc(C(=O)N[C@@H](C)Cn2ccc(-c3ccc(C#N)c(Cl)c3)n2)n[nH]1]

Darolutamide Form II of the present invention may be characterized by any one of the following embodiments or by combining two or more of the following embodiments.

Hence, in a first aspect the present invention relates to a crystalline form of darolutamide (Form II) which can be characterized by having a powder X-ray diffractogram comprising reflections at 2-Theta angles of:

(5.9±0.2)°, (9.1±0.2)° and (16.4±0.2)°; or
(5.9±0.2)°. (7.5±0.2)°. (9.1±0.2)° and (16.4±0.2)°; or
(5.9±0.2)°, (7.5±0.2)°, (9.1±0.2)°, (13.7±0.2)° and (16.4±0.2)°; or
(5.9±0.2)°, (7.5±0.2)°, (9.1±0.2)°, (13.7±0.2)°, (15.0±0.2)° and (16.4±0.2)°; or
(5.9±0.2)°, (7.5±0.2)°, (9.1±0.2)°, (11.8±0.2)°, (13.7±0.2)°, (15.0±0.2)° and (16.4±0.2)°; or
(5.9±0.2)°, (7.5±0.2)°, (9.1±0.2)°, (11.8±0.2)°, (13.7±0.2)°, (15.0±0.2)°, (16.4±0.2)° and (22.6±0.2)°; or
(5.9±0.2)°, (7.5±0.2)°, (9.1±0.2)°, (11.8±0.2)°, (13.7±0.2)°, (15.0±0.2)°, (16.4±0.2)°, (20.3±0.2)° and (22.6±0.2)°; or
(5.9±0.2)°, (7.5±0.2)°, (9.1±0.2)°, (11.8±0.2)°, (13.7±0.2)°, (15.0±0.2)°, (16.4±0.2)°, (18.2±0.2)°, (20.3±0.2)° and (22.6±0.2)°; or
(5.9±0.2)°, (7.5±0.2)°, (9.1±0.2)°, (11.8±0.2)°, (13.7±0.2)°, (15.0±0.2)°, (16.4±0.2)°, (18.2±0.2)°, (20.3±0.2)°, (22.6±0.2)° and (24.6±0.2)°; or
(5.9±0.2)°, (7.5±0.2)°, (9.1±0.2)°, (11.8±0.2)°, (13.7±0.2)°, (15.0±0.2)°, (16.4±0.2)°, (18.2±0.2)°, (20.3±0.2)°, (22.6±0.2)°, (23.5±0.2)° and (24.6±0.2)°; or
(5.9±0.2)°, (7.5±0.2)°, (9.1±0.2)°, (11.8±0.2)°, (13.7±0.2)°, (15.0±0.2)°, (16.4±0.2)°, (18.2±0.2)°, (20.3±0.2)°, (22.6±0.2)°, (23.5±0.2)°, (24.6±0.2)° and (27.8±0.2)°; or
(5.9±0.2)°, (7.5±0.2)°, (9.1±0.2)°, (11.8±0.2)°, (13.7±0.2)°, (15.0±0.2)°, (16.4±0.2)°, (18.2±0.2)°, (20.3±0.2)°, (22.6±0.2)°, (23.5±0.2)°, (24.6±0.2)°, (25.5±0.2)° and (27.8±0.2)°; or
(5.9±0.2)°, (7.5±0.2)°, (9.1±0.2)°, (11.8±0.2)°, (13.7±0.2)°, (15.0±0.2)°, (16.4±0.2)°, (18.2±0.2)°, (19.1±0.2)°, (20.3±0.2)°, (22.6±0.2)°, (23.5±0.2)°, (24.6±0.2)°, (25.5±0.2)° and (27.8±0.2)°; or
(5.9±0.2)°, (7.5±0.2)°, (9.1±0.2)°, (11.8±0.2)°, (13.7±0.2)°, (15.0±0.2)°, (16.4±0.2)°, (17.1±0.2)°, (18.2±0.2)°, (19.1±0.2)°, (20.3±0.2)°, (22.6±0.2)°, (23.5±0.2)°, (24.6±0.2)°, (25.5±0.2)° and (27.8±0.2)°; or
(5.9±0.2)°, (7.5±0.2)°, (9.1±0.2)°, (11.8±0.2)°, (13.7±0.2)°, (15.0±0.2)°, (16.4±0.2)°, (17.1±0.2)°, (17.8±0.2)°, (18.2±0.2)°, (19.1±0.2)°, (20.3±0.2)°, (22.6±0.2)°, (23.5±0.2)°, (24.6±0.2)°, (25.5±0.2)° and (27.8±0.2)°,
when measured at a temperature in the range of from 20 to 30° C. with Cu-Kalpha_{1,2} radiation having a wavelength of 0.15419 nm.

In another embodiment the present invention relates to a crystalline form of darolutamide (Form II) which can be characterized by having a powder X-ray diffractogram comprising reflections at 2-Theta angles of:

(5.9±0.1)°, (9.1±0.1)° and (16.4±0.1)°; or
(5.9±0.1)°, (7.5±0.1)°, (9.1±0.1)° and (16.4±0.1)° or
(5.9±0.1)°, (7.5±0.1)°, (9.1±0.1)°, (13.7±0.1)° and (16.4±0.1)°; or
(5.9±0.1)°, (7.5±0.1)°, (9.1±0.1)°, (13.7±0.1)°, (15.0±0.1)° and (16.4±0.1)°; or
(5.9±0.1)°, (7.5±0.1)°, (9.1±0.1)°, (11.8±0.1)°, (13.7±0.1)°, (15.0±0.1)° and (16.4±0.1)°; or
(5.9±0.1)°, (7.5±0.1)°, (9.1±0.1)°, (11.8±0.1)°, (13.7±0.1)°, (15.0±0.1)°, (16.4±0.1)° and (22.6±0.1)°; or
(5.9±0.1)°, (7.5±0.1)°, (9.1±0.1)°, (11.8±0.1)°, (13.7±0.1)°, (15.0±0.1)°, (16.4±0.1)°, (20.3±0.1)° and (22.6±0.1)°; or
(5.9±0.1)°, (7.5±0.1)°, (9.1±0.1)°, (11.8±0.1)°, (13.7±0.1)°, (15.0±0.1)°, (16.4±0.1)°, (18.2±0.1)°, (20.3±0.1)° and (22.6±0.1)°; or
(5.9±0.1)°, (7.5±0.1)°, (9.1±0.1)°, (11.8±0.1)°, (13.7±0.1)°, (15.0±0.1)°, (16.4±0.1)°, (18.2±0.1)°, (20.3±0.1)°, (22.6±0.1)° and (24.6±0.1)°; or
(5.9±0.1)°, (7.5±0.1)°, (9.1±0.1)°, (11.8±0.1)°, (13.7±0.1)°, (15.0±0.1)°, (16.4±0.1)°, (18.2±0.1)°, (20.3±0.1)°, (22.6±0.1)°, (23.5±0.1)° and (24.6±0.1)°; or
(5.9±0.1)°, (7.5±0.1)°, (9.1±0.1)°, (11.8±0.1)°, (13.7±0.1)°, (15.0±0.1)°, (16.4±0.1)°, (18.2±0.1)°, (20.3±0.1)°, (22.6±0.1)°, (23.5±0.1)°, (24.6±0.1)° and (27.8±0.1)°; or
(5.9±0.1)°, (7.5±0.1)°, (9.1±0.1)°, (11.8±0.1)°, (13.7±0.1)°, (15.0±0.1)°, (16.4±0.1)°, (18.2±0.1)°, (20.3±0.1)°, (22.6±0.1)°, (23.5±0.1)°, (24.6±0.1)°, (25.5±0.1)° and (27.8±0.1)°; or
(5.9±0.1)°, (7.5±0.1)°, (9.1±0.1)°, (11.8±0.1)°, (13.7±0.1)°, (15.0±0.1)°, (16.4±0.1)°, (18.2±0.1)°, (19.1±0.1)°, (20.3±0.1)°, (22.6±0.1)°, (23.5±0.1)°, (24.6±0.1)°, (25.5±0.1)° and (27.8±0.1)°; or
(5.9±0.1)°, (7.5±0.1)°, (9.1±0.1)°, (11.8±0.1)°, (13.7±0.1)°, (15.0±0.1)°, (16.4±0.1)°, (17.1±0.1)°, (18.2±0.1)°, (19.1±0.1)°, (20.3±0.1)°, (22.6±0.1)°, (23.5±0.1)°, (24.6±0.1)°, (25.5±0.1)° and (27.8±0.1)°; or
(5.9±0.1)°, (7.5±0.1)°, (9.1±0.1)°, (11.8±0.1)°, (13.7±0.1)°, (15.0±0.1)°, (16.4±0.1)°, (17.1±0.1)°, (17.8±0.1)°, (18.2±0.1)°, (19.1±0.1)°, (20.3±0.1)°, (22.6±0.1)°, (23.5±0.1)°, (24.6±0.1)°, (25.5±0.1)° and (27.8±0.1)°,
when measured at a temperature in the range of from 20 to 30° C. with Cu-Kalpha_{1,2} radiation having a wavelength of 0.15419 nm.

The powder X-ray diffractogram of Form II of darolutamide of the present invention is readily distinguishable from the known Form I of darolutamide of WO 2016/120530 A1 (see also the PXRD overlay displayed in FIG. 6 herein). Form II shows for example reflections at 2-Theta angles of (5.9±0.2)°, (7.5±0.2)° and (9.1±0.2)°, whereas Form I shows no reflection in these ranges. On the other hand, Form II shows no reflections at 2-Theta angles of (8.5±0.2)° and (10.4±0.2)°, whereas Form I possesses very characteristic, namely the most intensive reflections in these ranges. Thus, in another aspect, the present invention relates to a crystalline form of darolutamide (Form II) which can be characterized by having a powder X-ray diffractogram comprising reflections at 2-Theta angles as described above, but comprising no reflections at 2-Theta angles of (8.5±0.2)° and (10.4±0.2)°, when measured at a temperature in the range of from 20 to 30° C. with Cu-Kalpha_{1,2} radiation having a wavelength of 0.15419 nm.

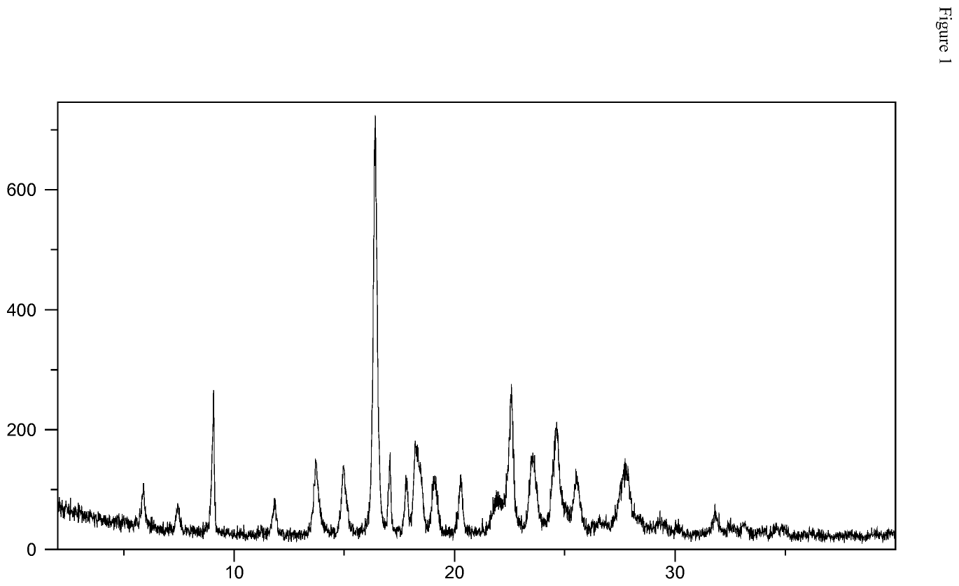

In yet another embodiment, the present invention relates to a crystalline form of darolutamide (Form II) characterized by having a powder X-ray diffractogram essentially the same as shown in FIG. 1 of the present invention, when measured at a temperature in the range of from 20 to 30° C. with Cu-Kalpha_{1,2} radiation having a wavelength of 0.15419 nm.

In a further embodiment, the present invention relates to a crystalline form of darolutamide (Form II) characterized by having a FTIR spectrum comprising peaks at wavenumbers of:

(3406±2) cm⁻¹, (3252±2) cm⁻¹ and (3165±2) cm⁻¹ or;
(3406±2) cm⁻¹, (3252±2) cm⁻¹, (3165±2) cm⁻¹ and (2233±2) cm⁻¹; or
(3406±2) cm⁻¹, (3252±2) cm⁻¹, (3165±2) cm⁻¹, (2233±2) cm⁻¹ and (1647±2) cm⁻¹; or
(3406±2) cm⁻¹, (3252±2) cm⁻¹, (3165±2) cm⁻¹, (2233±2) cm⁻¹, (1647±2) cm⁻¹ and (1539±2) cm⁻¹; or
(3406±2) cm⁻¹, (3252±2) cm⁻¹, (3165±2) cm⁻¹, (2233±2) cm⁻¹, (1647±2) cm⁻¹, (1601±2) cm⁻¹ and (1539±2) cm⁻¹; or
(3406±2) cm⁻¹, (3252±2) cm⁻¹, (3165±2) cm⁻¹, (2980±2) cm⁻¹, (2233±2) cm⁻¹, (1647±2) cm⁻¹, (1601±2) cm⁻¹ and (1539±2) cm⁻¹; or
(3406±2) cm⁻¹, (3252±2) cm⁻¹, (3165±2) cm⁻¹, (2980±2) cm⁻¹, (2233±2) cm⁻¹, (1647±2) cm⁻¹, (1601±2) cm⁻¹, (1539±2) cm⁻¹ and (760±2) cm⁻¹; or
(3406±2) cm⁻¹, (3252±2) cm⁻¹, (3165±2) cm⁻¹, (2980±2) cm⁻¹, (2233±2) cm⁻¹, (1647±2) cm⁻¹, (1601±2) cm⁻¹, (1539±2) cm⁻¹, (760±2) cm⁻¹ and (694±2) cm⁻¹,
when measured at a temperature in the range of from 20 to 30° C. with a diamond ATR cell.

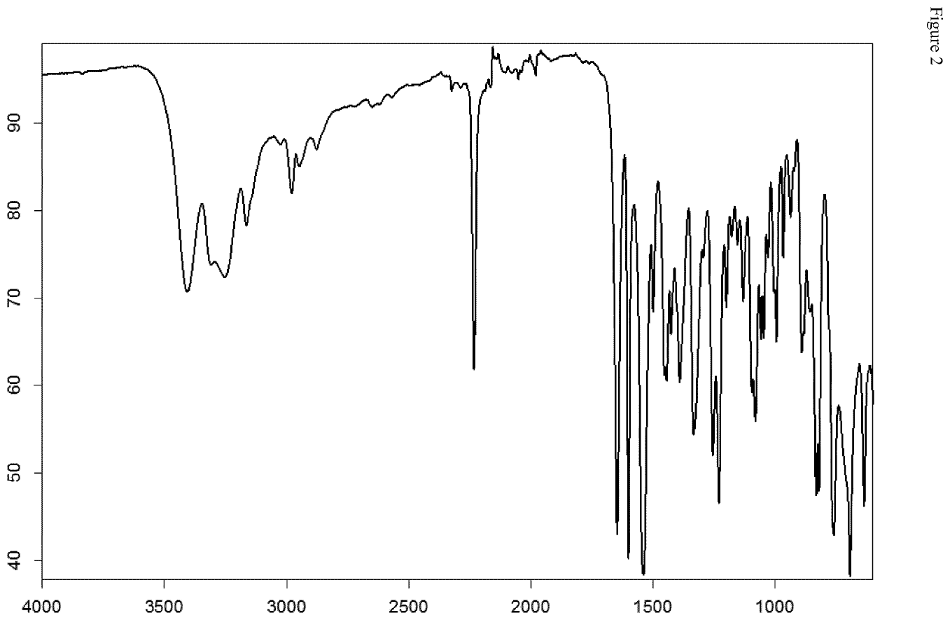

In yet another embodiment, the present invention relates to a crystalline form of darolutamide (Form II) characterized by having a FTIR spectrum essentially the same as shown in FIG. 2 of the present invention, when measured at a temperature in the range of from 20 to 30° C. with a diamond ATR cell.

In another embodiment, the present invention relates to a crystalline form of darolutamide (Form II) characterized by having a DSC curve comprising an endothermic peak, preferably a single endothermic peak, having an onset temperature of (167±1)° C., when measured with DSC at a heating rate of 10 K/min.

In a further embodiment, the present invention relates to a crystalline form of darolutamide (Form II) characterized by having a DSC curve comprising an endothermic peak, preferably a single endothermic peak, having a peak temperature of (169±1)° C., when measured with DSC at a heating rate of 10 K/min.

In another embodiment, the present invention relates to a crystalline form of darolutamide (Form II), characterized by having a TGA curve showing a mass loss of 0.7 weight %, based on the weight of the crystalline form, when measured in the range of from 25 to 170° C. at a rate of 10 K/min.

In still another embodiment, the present invention relates to a crystalline form of darolutamide (Form II) characterized in that the crystalline form is anhydrous.

In yet another embodiment, the present invention relates to a crystalline form of darolutamide (Form II) characterized in that the crystalline form is non-solvated.

In a further embodiment, the present invention relates to a crystalline form of darolutamide (Form II) comprising lath-shaped and/or needle-shaped crystals. Preferably, the lath and/or needle shaped crystals are agglomerated if form of spherical particles.

In a further aspect, the present invention relates to a composition comprising Form II of darolutamide of the present invention as defined according to any one of the embodiments herein above, which is essentially free of any other physical forms of darolutamide. For example, a composition comprising Form II of darolutamide of the present invention comprises at most 20 weight %, preferably at most 10 weight %, more preferably at most 5 weight %, even more preferably at most 2 weight % and most preferably at most 1 weight % of any other physical form of darolutamide, based on the weight of the composition. Preferably, the any other physical form of darolutamide is Form I of WO 2016/120530 A1 or amorphous. In a further preferred embodiment, the invention relates to a composition comprising Form II of darolutamide as defined according to any one of the embodiments herein above, characterized by having a PXRD comprising no reflections at 2-Theta angles of (8.5±0.2)° and (10.4±0.2)°, when measured at a temperature in the range of from 20 to 30° C. with Cu-Kalpha_{1,2} radiation having a wavelength of 0.15419 nm.

In another aspect, the present invention relates to a process for the preparation of crystalline Form II of darolutamide of the present invention or the composition comprising Form II of darolutamide as defined above comprising:
(a) dissolving darolutamide in a solvent selected from the group consisting of methanol, 2-propanol, n-butanol, tert-amylalcohol, acetone, methyl acetate, 1,4-dioxane, dimethylformamide and acetic acid or any mixture thereof;
(b) optionally filtering the solution obtained in step (a)
(c) adding an antisolvent selected from an alkane or water to the solution obtained in step (a) or (b) or vice versa, characterized in that the temperature of the mixture is maintained in the range of from 20 to 30° C. during the addition;
(d) optionally separating at least a part of the crystals obtained in step (c) from their mother liquor;
(e) optionally washing the isolated crystals obtained in step (d);
(f) optionally drying the crystals obtained in any one of steps (c) to (e);

Darolutamide may be prepared according to the teaching of WO 2011/051540 A1, in particular according to the teaching of Example 56 therein. In step (a) of the above described process at least part, preferably all of darolutamide is dissolved in a solvent, wherein the solvent is selected from the group consisting of methanol, 2-propanol, n-butanol, tert-amylalcohol, acetone, methyl acetate, 1,4-dioxane, dimethylformamide and acetic acid or any mixture thereof. Preferably, n-butanol is used as solvent. The solution is either prepared at room temperature or, preferably, by heating the mixture comprising darolutamide and the applied solvent e.g. to the reflux temperature of the solvent used. For example, if n-butanol is used as solvent the mixture may be heated from about 80 to 118° C. during the dissolution step. The exact temperature needed for dissolution also depends on the applied darolutamide concentration. The darolutamide concentration of the solution in step (a) usually ranges from about 20 to 50 g/L, preferably from about 30 to 40 g/L, for example when n-butanol is used as solvent, the darolutamide concentration of the solution may be in the range of from 30 to 35 g/L, such as 34 g/L. Optionally, the solution may be filtered in order to remove any potentially present non-dissolved particles and to obtain a clear solution.

Then, the temperature of the solution is decreased to a temperature in the range of from about 20 to 30° C. Once the mixture has reached said temperature, antisolvent is added to the solution in order to initiate crystallization, whereat the antisolvent addition rate is not critical. In one embodiment, the darolutamide solution obtained in step (a) or (b) of the above described process may be added to the antisolvent. Suitable antisolvents which may be used are selected from alkanes or water, wherein suitable alkanes may be selected from the group consisting of n-pentane, n-hexane, cyclohexane or n-heptane or any mixtures thereof and most preferably n-heptane is used. When combining the darolutamide solution with the antisolvent, it is important that the temperature of the mixture is kept in the range of from about 20 to 30° C. in order to ensure the nucleation and crystallization of crystalline Form II of the present invention. The applied solvent/antisolvent ratio usually ranges from about 1:3 to 1:40, preferably from about 1:3 to 1:10, more preferably from about 1:4 to 1:5. Optionally, Form II seed crystals may be added in order to promote crystallization and/or to control the particle size distribution of the finally obtained material. The seed crystal amount may range from about 1 to 10 weight % based on the amount of darolutamide present in the solution. After having combined the solution obtained in step (a) or (b) with the antisolvent, the obtained mixture is preferably further stirred at a temperature in the range of from about 20 to 30° C. until plentiful crystallization, which is indicated by the presence of a homogenous suspension. Usually, the mixture is further stirred for a period ranging from about 1 to 48 hours, preferably from about 1 to 24 hours and most preferably from about 1 to 12 hours such as 4 to 6 hours.

Optionally, in the next step, at least a part of the crystals are separated from their mother liquor. Preferably, the crystals are separated from their mother liquor by any conventional method such as filtration, centrifugation, solvent evaporation or decantation, more preferably by filtration or centrifugation and most preferably by filtration.

In a further optional step, the isolated crystals may be washed with a suitable liquid, for example, the isolated crystals may be washed with the same antisolvent used for initiating the crystallization.

The crystals obtained from any one of steps (c) to (e), preferably from step (d) or (e) are subsequently dried, wherein drying is performed at a temperature in the range of from about 20 to 60° C., preferably in the range of from about 20 to 30° C. and a vacuum in the range of from about 1 to 50 mbar, for example in the range of from about 10 to 30 mbar. Typically, drying is performed for a period in the range of from about 6 to 72 hours, preferably from 10 to 48 hours, more preferably from 12 to 24 hours.

As already mentioned above darolutamide Form II of the present invention combines excellent powder properties with high solubility and is therefore the preferred polymorph of darolutamide for the preparation of a pharmaceutical composition.

For example, the PXRD overlay of Form I and Form II, which is displayed in FIG. 6 herein indicates that Form II of the present invention shows higher crystallinity than Form I WO 2016/120530 A1. Form I used for this overlay was prepared according to the teaching of Example 1 of WO 2016/120530 A1 and both diffractograms were collected using the same measurement conditions and equipment as outlined in Example 1 herein. As can be seen from FIG. 6, the diffractogram on the bottom belonging to Form II of the present invention shows sharp and well-defined reflections and a straight baseline, indicating high crystallinity and the absence of amorphous material. In contrast, the diffractogram on the top, which belongs to Form I shows very broad and undefined reflections at 2-Theta angles above about 15°. Furthermore, also in the region above about 15° 2-Theta a halo is visible in the background. Both indicates a low degree of crystallinity and the presence of amorphous material. In FIG. 1 of WO 2016/120530 A1 and in FIG. 1 of CN107602471 the halo in the Form I diffractogram is even more pronounced and again very broad reflections are present. WO 2016/120530 A1 and CN107602471 are using different crystallization processes for the preparation of Form I. This suggests that the low crystallinity of Form 1 appears regardless of the crystallization procedure and thus is an intrinsic property of this particular form.

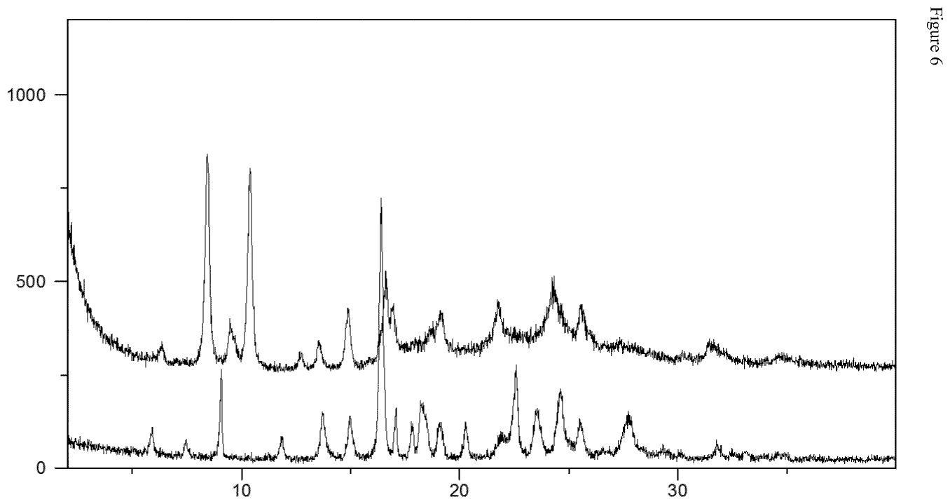

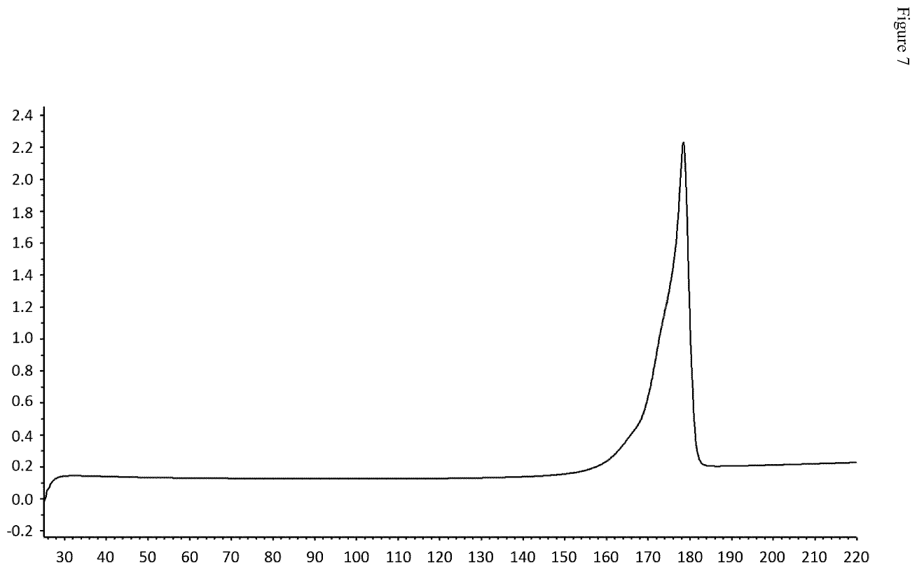

The low crystallinity of Form I is also indicated by a very broad melting peak in the DSC curve of Form I, which is displayed in FIG. 7 herein. Form I used for this DSC experiment was prepared according to the teaching of Example 1 of WO 2016/120530 A1 and the DSC thermogram was collected using the same measurement conditions and equipment as outlined in Example 1 herein. On the other hand Form II of the present invention shows a significantly sharper melting peak, which is a consequence of the higher degree of crystallinity of Form II.

A polymorph of low crystallinity and/or contaminated with an amorphous phase is not desired for pharmaceutical development since such a material is prone to phase changes e.g. crystallization during pharmaceutical processing and/or storage and thus may lead to non-uniform drug products. In addition, such material usually shows bad processability because it is often electrically charged and/or hygroscopic, shows poor flowability, compactibility and/or wettability.

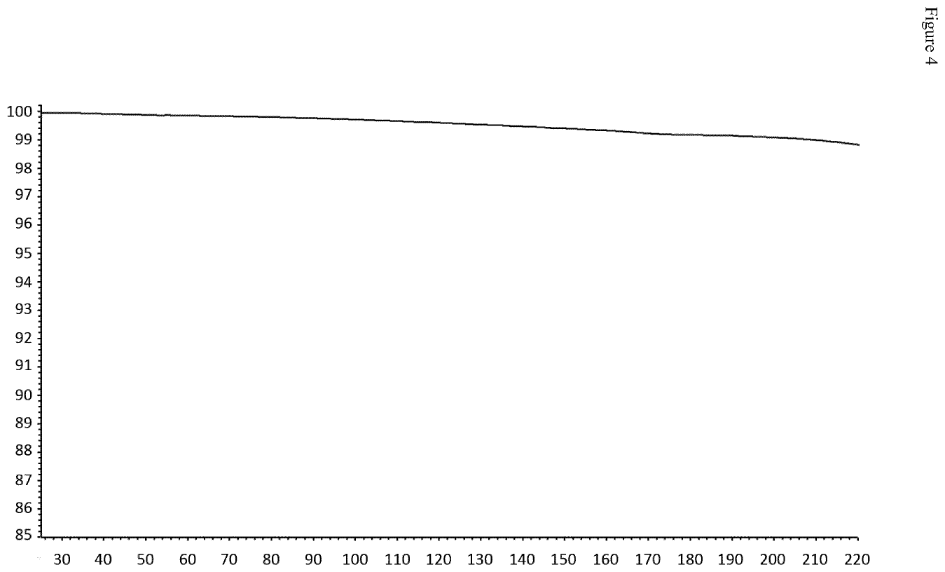

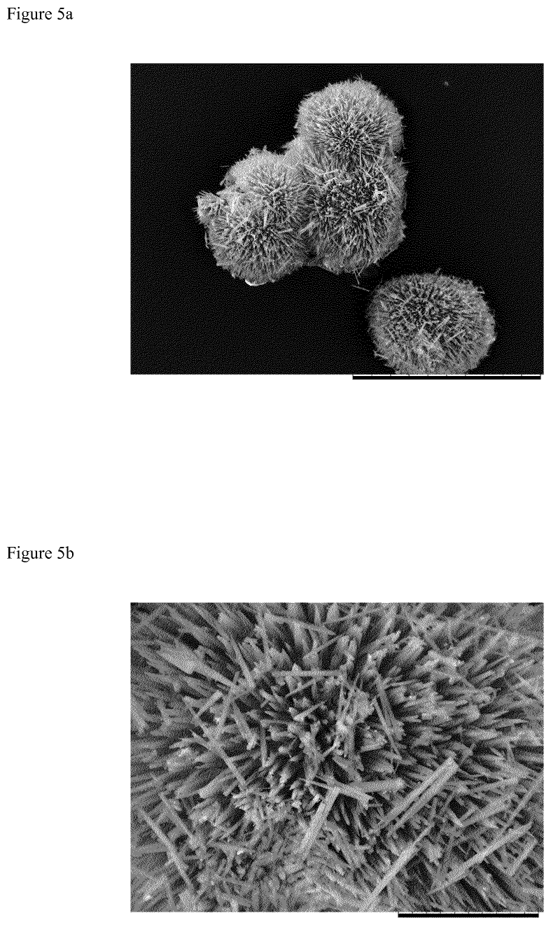

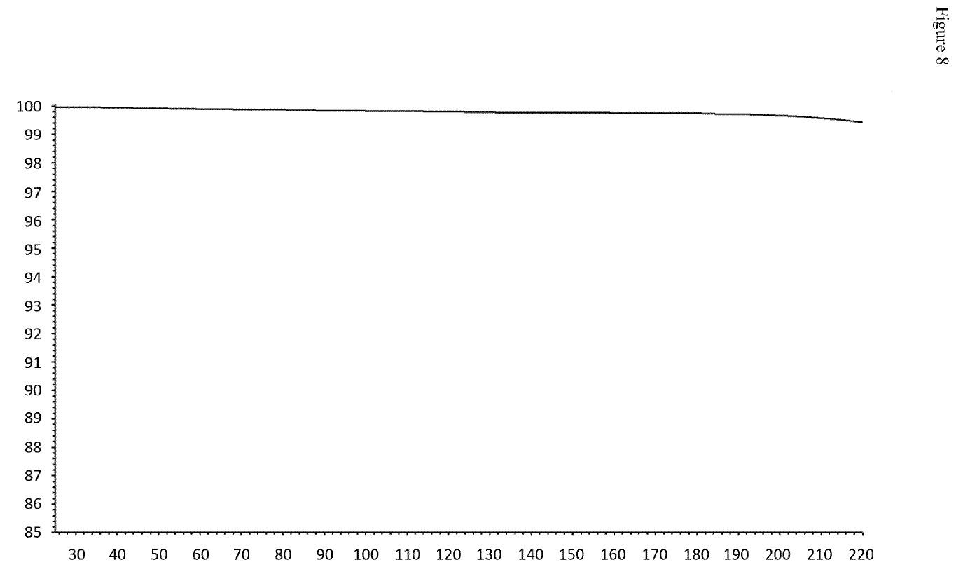

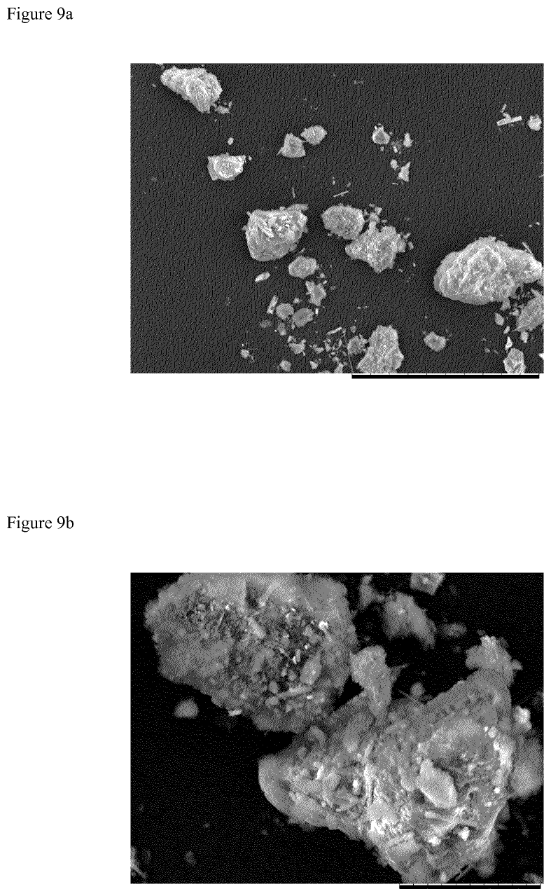

The poor powder properties of Form I result also from the undefined and heterogenous morphology of the Form I crystals, which are randomly agglomerated as can be seen in the scanning electron microscopic images displayed in FIGS. 9a and 9b herein. On the other hand, the excellent flow properties of Form II of the present invention can be explained by the well defined needle-shaped to lath-shaped morphology of the Form II crystals, which are arranged in spheres (see FIGS. 5a and 5b herein).

Furthermore, darolutamide Form II of the present invention possesses increased solubility in physiologically relevant aqueous media compared to Form I of WO 2016/120530 A1, which is a crucial advantage for a low soluble drug substance like darolutamide in order to reach the desired blood levels. The present inventors have also found that darolutamide Form II of the present invention shows a significantly lower food effect compared to darolutamide Form I, and surprisingly darolutamide form II of the present invention can even be administered without food, while still achieving therapeutically effective blood levels. When darolutamide form II is administered with food—a requirement for form I if therapeutically effective blood levels are to be achieved with a dosing regimen of 2×300 mg tablets twice daily—the amount to be used can be lowered by 10% to 70% compared to darolutamide form I.

In summary, Form II of darolutamide of the present invention shows improved properties when compared with Form I and is therefore, for example, useful to enhance the processability of darolutamide during drug product manufacture and to enhance drug product bioavailability.

In a further aspect, the invention relates to crystalline Form II of darolutamide of the present invention or the composition comprising the same as defined above for use in the treatment and/or prophylaxis of cancer, in particular for use in the treatment of androgen dependent cancer, such as prostate cancer for example castration-resistant prostate cancer (CRPC) and metastatic hormone-sensitive prostate cancer (HSPC). In a specific embodiment, the invention relates to the use of crystalline Form II of darolutamide of the present invention or the composition comprising the same as defined above in the treatment and/or prophylaxis of cancer, in particular androgen dependent cancer, such as prostate cancer for example castration-resistant prostate cancer (CRPC) and metastatic hormone-sensitive prostate cancer (HSPC), characterized in that crystalline Form II of darolutamide of the present invention or the composition comprising the same as defined above is administered at a daily dose selected from the group consisting of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1800 mg, 1850 mg, 1900 mg, 1950 mg and 2000 mg, more preferably at a daily dose of 600 mg or 1200 mg, calculated as darolutamide, to a patient in need of such a treatment or prophylaxis.

In yet another aspect, the present invention is directed to a method of treating or prophylactically preventing cancer in particular androgen dependent cancer, such as prostate cancer for example castration-resistant prostate cancer (CRPC) and metastatic hormone-sensitive prostate cancer (HSPC) comprising administering to a patient in need thereof the crystalline Form II of darolutamide of the present invention or the composition comprising the same as defined above. Preferably, the crystalline Form II of darolutamide, or the composition comprising the same as defined above is administered at a daily dose selected from the group consisting of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1800 mg, 1850 mg, 1900 mg, 1950 mg and 2000 mg, more preferably at a daily dose of 600 mg or 1200 mg, calculated as darolutamide, to a patient in need of such a treatment or prophylaxis.

A preferred daily dose for administration with food is from 360 mg to 1080 mg darolutamide form II, such as from 500 mg to 1000 mg, for example from 600 mg to 900 mg. It has surprisingly been found that such a daily dose of darolutamide form II can provide blood levels of darolutamide which are equivalent to a daily dose of 1200 mg darolutamide form I under fed conditions.

A preferred daily dose for administration without food, such as under fasted conditions, is also from 360 mg to 1080 mg darolutamide form II, such as from 500 mg to 1000 mg, for example from 600 mg to 900 mg. It has surprisingly been found that such a daily dose of darolutamide form II can provide blood levels of darolutamide which are equivalent to a daily dose of 1200 mg darolutamide form I.

The fact that darolutamide form II can also achieve therapeutically effective blood levels when administered in the fasted state is a major improvement over form I, which needs administration with food. For example, the treatment regimen in patients with co-morbidities, which require medication that need administration in the fasted state, is greatly facilitated.

Preferably, the crystalline Form II of darolutamide of the present invention or the composition comprising the same as defined above is formulated into a pharmaceutical composition.

Hence, in a further aspect, the present invention relates to the use of crystalline Form II of darolutamide of the present invention or the composition comprising the same as defined above for the preparation of a pharmaceutical composition.

In a further aspect, the present invention relates to a pharmaceutical composition comprising crystalline Form II of darolutamide or the composition comprising the same as defined above, preferably in an effective and/or predetermined amount, and at least one pharmaceutically acceptable excipient. Most preferably, the pharmaceutical composition of the present invention is an oral solid dosage form, such as a tablet or a capsule. Preferably, the pharmaceutical composition of the present invention is a tablet, most preferably a film-coated tablet. In one embodiment, the tablet is film-coated with a coating material comprising polyvinyl alcohol (e.g. partially hydrolyzed), iron oxide, talc, and titanium dioxide. In another embodiment the coating material additionally comprises polyethylene glycol.

The at least one pharmaceutically acceptable excipient, which is comprised in the pharmaceutical composition of the present invention, is preferably selected from the group consisting of carriers, fillers, diluents, lubricants, sweeteners, stabilizing agents, solubilizing agents, antioxidants and preservatives, flavouring agents, binders, colorants, osmotic agents, buffers, surfactants, disintegrants, granulating agents, coating materials and combinations thereof. More preferably, the at least one pharmaceutically acceptable excipient is selected from the group consisting of fillers, diluents, lubricants, disintegrants and coating materials. In one embodiment all of the latter pharmaceutically acceptable excipients are comprised by the pharmaceutical composition of the present invention.

In a preferred embodiment, the at least one pharmaceutically acceptable excipient is selected from the group consisting of microcrystalline cellulose, croscarmellose sodium and magnesium stearate. In a preferred embodiment, all of the latter pharmaceutically acceptable excipients are comprised by the pharmaceutical composition of the present invention.

Preferably, the present invention relates to a pharmaceutical composition as describe above, wherein the predetermined and/or effective amount of the crystalline Form II of darolutamide or the composition comprising the same as defined above is selected from the group consisting of 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg and 1200 mg, preferably from 300 mg, 600 mg and 1200 mg and most preferably from 300 mg and 600 mg, calculated as darolutamide. Preferred predetermined and/or effective amounts of the crystalline Form II of darolutamide or the composition comprising the same as defined above per single oral dosage form are from 180 mg to 540 mg, such as from 250 mg to 500 mg, for example from 300 mg to 450 mg.

Preferably, the present invention relates to a pharmaceutical composition as described above, wherein the pharmaceutical composition is administered once-daily or twice-daily.

In a further aspect, the present invention relates to the pharmaceutical composition as described above for use as a medicament.

In yet another aspect, the present invention relates to the pharmaceutical composition as described above for use in the treatment or prophylaxis of cancer, in particular androgen dependent cancer, such as prostate cancer for example castration-resistant prostate cancer (CRPC) and metastatic hormone-sensitive prostate cancer (HSPC).

In another embodiment, the present invention is directed to a method of treating or prophylactically preventing cancer, in particular androgen dependent cancer, such as prostate cancer for example castration-resistant prostate cancer (CRPC) and metastatic hormone-sensitive prostate cancer (HSPC) by administering the pharmaceutical composition as described above to a patient in need of such a treatment and/or prophylaxis.

In a second concept, the present inventors have found an improved dosage regimen for oral solid dosage forms comprising darolutamide form I. In clinical trials 2×300 mg tablets were used, presumably because tablets comprising darolutamide form I as obtainable from WO 2016/120530A1 in high drug load have poor dissolution behavior. WO2018/162793A1 discloses that crystalline particles of darolutamide having a volume median diameter between 10-1000 μm have rounded particle shape and are easy to isolate, free flowing and exhibit reduced stickiness.

The present invention provides tablets having a high drug load of darolutamide such that instead of 2×2 oral solid dosage forms per day only 2×1 oral solid dosage forms are to be administered per day, resulting in reduced pill burden for the patient. The inventors have found that fast dissolving tablets comprising a pharmaceutically effective amount of darolutamide in an amount of from 50% to 80% in weight based on the total weight of the tablet can be obtained when a) crystalline darolutamide form II is used as the active pharmaceutical ingredient or b) when crystalline darolutamide form I particles characterized by a specific particle size distribution are used as the active pharmaceutical ingredient.

The present invention therefore also relates to a tablet comprising
a) at least one pharmaceutically acceptable excipient suitable for the preparation of tablets, and
   b1) crystalline darolutamide form II, or
   b2) crystalline darolutamide form I in the form of particles characterized by having a particle size distribution wherein d(50)=20-150 μm and/or d(90)=150 μm-300 μm, more preferably by d(50)=30-150 μm and/or d(90)=150-200 μm,
   wherein the amount of darolutamide, calculated as the percentage of the content in weight of darolutamide based on the total weight of the tablet, is from 50 percent to 90 percent, e.g. at least about 55, 60 or 65 percent, to 80, 85 or 90 percent, preferably more than 60 percent. In particular the amount of darolutamide may vary from 60 to 90 percent, e.g. from 65 to 85 percent in weight of darolutamide based on the total weight of the tablet.
   One or more pharmaceutically acceptable excipients may be present in the tablets of the second concept, e.g. those conventionally used, e.g. (a.1) at least one binder, e.g. microcrystalline cellulose, hydroxypropylmethyl cellulose, (a.2) at least one disintegrant, e.g. cross-linked polyvinylpyrrolidinone, e.g. Crospovidone®, (a.3) at least one glidant, e.g. colloidal silicon dioxide, (a.4) at least one lubricant, e.g. magnesium stearate and/or (a.5) basic coating. In the tablet according to the present invention, microcrystalline cellulose is used as a binder.
   Tablets according to the second concept surprisingly provide for the administration of darolutamide in tablets of a smaller size than was hitherto possible for a given unit dose of darolutamide. The tablets of the second concept are, despite the high drug loading, small, and, therefore, convenient to administer. This leads to a better patient compliance. In a preferred embodiment this invention provides a high drug load tablet as described in the second concept comprising from 400 mg to 900 mg darolutamide form I, e.g. of from 500 mg to 800 mg, such as about 600 mg.
   Alternatively this invention provides a high drug load tablet as described in the second concept comprising from 180 mg to 540 mg darolutamide form II, e.g. of from 250 mg to 500 mg, for example from 300 mg to 450 mg.
   Darolutamide form I having the desired particle size distribution can be obtained by fractionated sieving after gentle compression of darolutamide form I obtainable from WO 2016/120530A1. Harsh milling conditions, such as encountered during prolonged ball milling, should be avoided during size reduction of darolutamide form I since darolutamide form I can then transform to the amorphous state.

The present invention is further illustrated by the following embodiments and combinations of embodiments resulting from the given dependencies and back-references:
1) A crystalline form of N-{(2S)-1-[3-(3-chloro-4-cyanophenyl)-1H-pyrazol-1-yl]-propan-2-yl}-5-[(1RS)-1-hydroxyethyl]-1H-pyrazole-3-carboxamide (Form II) characterized by having a powder X-ray diffractogram comprising reflections at 2-Theta angles of (5.9±0.2)°, (9.1±0.2)° and (16.4±0.2)°, when measured at a temperature in the range of from 20 to 30° C. with Cu-Kalpha_{1,2} radiation having a wavelength of 0.15419 nm.
2) The crystalline form of item 1 characterized by having a powder X-ray diffractogram comprising additional reflections at 2-Theta angles of (7.5±0.2)° and (13.7±0.2)°, when measured at a temperature in the range of from 20 to 30° C. with Cu-Kalpha_{1,2} radiation having a wavelength of 0.15419 nm.
3) The crystalline form of item 1 or 2 characterized by having a powder X-ray diffractogram comprising no reflections at 2-Theta angles of (8.5±0.2)° and (10.4±0.2)°, when measured at a temperature in the range of from 20 to 30° C. with Cu-Kalpha_{1,2} radiation having a wavelength of 0.15419 nm.
4) A crystalline form of N-{(2S)-1-[3-(3-chloro-4-cyanophenyl)-1H-pyrazol-1-yl]-propan-2-yl}-5-[(1RS)-1-hydroxyethyl]-1H-pyrazole-3-carboxamide (Form II) characterized by having a Fourier transform infrared spectrum comprising peaks at wavenumbers of (3406±2) cm⁻¹, (3252±2) cm⁻¹ and (3165±2) cm⁻¹, when measured at a temperature in the range of from 20 to 30° C. with a diamond attenuated total reflection cell.
5) A crystalline form of darolutamide characterized by having a DSC curve comprising an endothermic peak, preferably a single endothermic peak, having an onset temperature of (167±1)° C., when measured with DSC at a heating rate of 10 K/min.
6) A crystalline form of darolutamide characterized by having a TGA curve showing a mass loss of 0.7 weight %, based on the weight of the crystalline form, when measured in the range of from 25 to 170° C. at a rate of 10 K/min.
7) The crystalline form of darolutamide according to any one of items 1 to 3, further characterized by the features of item 4.
8) The crystalline form of darolutamide according to any one of items 1 to 3, further characterized by the features of item 5.
9) The crystalline form of darolutamide according to any one of items 1 to 3, further characterized by the features of item 6.
10) The crystalline form of darolutamide according to any one of items 1 to 3, further characterized by the features of items 4 and 5, or by the features of items 4 and 6 or by the features of items 5 and 6.
11) The crystalline form of darolutamide according to item 4, further characterized by the features of any one of items 5 and 6, preferably by the features of both items 5 and 6.
12) The crystalline form according to any one of the preceeding items, wherein the crystalline form is anhydrous.
13) The crystalline form according to any one of the preceeding items, wherein the crystalline form is non-solvated.
14) The crystalline form according to any one of the preceeding items, wherein the crystalline form comprises lath-shaped and/or needle-shaped crystals.
15) The crystalline form according to item 14, wherein the lath and/or needle shaped crystals are agglomerated in the form of spherical particles.
16) A composition comprising the crystalline form according to any one of the preceding items and at most 5 weight % of any other physical form of N-{(2S)-1-[3-(3-chloro-4-cyanophenyl)-1H-pyrazol-1-yl]-propan-2-yl}-5-[(1RS)-1-hydroxyethyl]-1H-pyrazole-3-carboxamide, based on the weight of the composition.
17) The composition according to item 16, wherein the other physical form N-{(2S)-1-[3-(3-chloro-4-cyanophenyl)-1H-pyrazol-1-yl]-propan-2-yl}-5-[(1RS)-1-hydroxyethyl]-1H-pyrazole-3-carboxamide is Form I characterized by having a powder X-ray diffractogram comprising reflections at 2-Theta angles of (8.5±0.2)°, (10.4±0.2)°, (16.6±0.2)°, (16.9±0.2)° and (24.3±0.2)°, when measured at a temperature in the range of from 20 to 30° C. with Cu-Kalpha_{1,2} radiation having a wavelength of 0.15419 nm.
18) A process for the preparation of the crystalline form as defined in any one of items 1 to 15 or the composition as defined in items 16 or 17 comprising:
   (i) dissolving darolutamide in a solvent selected from the group consisting of methanol, 2-propanol, n-butanol, tert-amylalcohol, acetone, methyl acetate, 1,4-dioxane, dimethylformamide and acetic acid or any mixture thereof;
   (ii) adding an antisolvent selected from an alkane or water to the solution obtained in step (i) or vice versa, characterized in that the temperature of the mixture is maintained in the range of from 20 to 30° C. during the addition.
19) Use of the crystalline form as defined in any one of items 1 to 15 or the composition as defined in items 16 or 17 for the preparation of a pharmaceutical composition.
20) A pharmaceutical composition comprising the crystalline form as defined in any one of items 1 to 15 or the composition as defined in items 16 or 17, and at least one pharmaceutically acceptable excipient.
21) The pharmaceutical composition of item 20, wherein the pharmaceutical composition is an oral solid dosage form.
22) The pharmaceutical composition of item 21, wherein the oral solid dosage form is a tablet or a capsule.
23) The pharmaceutical composition of item 22 which is a film-coated tablet.
24) The pharmaceutical composition of item 23, wherein the tablet is film-coated with a coating material comprising polyvinyl alcohol (e.g. partially hydrolyzed), iron oxide, talc, and titanium dioxide.
25) The pharmaceutical composition of item 24 wherein the coating material additionally comprises polyethylene glycol.
26) The pharmaceutical composition according to any one of items 20 to 25, wherein the at least one pharmaceutically acceptable excipient is selected from the group consisting of carriers, fillers, diluents, lubricants, sweeteners, stabilizing agents, solubilizing agents, antioxidants and preservatives, flavouring agents, binders, colorants, osmotic agents, buffers, surfactants, disintegrants, granulating agents, coating materials and combinations thereof.
27) The pharmaceutical composition according to any one of items 20 to 25, wherein the at least one pharmaceutically acceptable excipient is selected from the group consisting of fillers, diluents, lubricants, disintegrants and coating materials.
28) The pharmaceutical composition according to any one of items 20 to 25, comprising all of the pharmaceutically acceptable excipients selected from the group consisting of fillers, diluents, lubricants, disintegrants and coating materials.
29) The pharmaceutical composition according to any one of items 20 to 28 for use as a medicament.
30) The pharmaceutical composition according to any one of items 20 to 29 for use in the treatment and/or prophylaxis of prostate cancer.
31) The pharmaceutical composition according to any one of items 20 to 30, wherein from 180 mg to 540 mg darolutamide form II are to be administered twice daily under fed conditions such that the total daily dose of darolutamide form II is from 360 mg to 1080 mg.
32) The pharmaceutical composition according to item 31, wherein from 250 mg to 500 mg darolutamide form II are to be administered twice daily under fed conditions such that the total daily dose of darolutamide form II is from 500 mg to 1000 mg.
33) The pharmaceutical composition according to any one of items 20 to 30, wherein from 180 mg to 540 mg darolutamide form II are to be administered twice daily under fasted conditions such that the total daily dose of darolutamide form II is from 360 mg to 1080 mg.
34) The pharmaceutical composition according to item 33, wherein from 250 mg to 500 mg darolutamide form II are to be administered twice daily under fed conditions such that the total daily dose of darolutamide form II is from 500 mg to 1000 mg.
35) A tablet comprising
   a) at least one pharmaceutically acceptable excipient suitable for the preparation of tablets, and
   b1) crystalline darolutamide form II, or
   b2) crystalline darolutamide form I in the form of particles characterized by having a particle size distribution wherein d(50)=20-150 μm and/or d(90)=150 μm-300 μm, and wherein the amount of darolutamide, calculated as the percentage of the content in weight of darolutamide based on the total weight of the tablet, is from 50 percent to 90 percent.
36) The tablet according to item 35 b2), wherein the particle size distribution is characterized by a d(50)=30-150 μm and/or d(90)=150-200 μm.
37) The tablet according to any one of items 35 or 36, wherein the darolutamide content is from 55 percent to 90 percent based on the total weight of the tablet.
38) The tablet according to item 37, wherein the darolutamide content is at least 60 percent.
39) The tablet according to item 38, wherein the darolutamide content is from 65 to 85 percent based on the total weight of the tablet.
40) The tablet according to any one of the items 35 to 39, wherein the at least one or more pharmaceutically acceptable excipient a) is selected from (a.1) at least one binder, e.g. microcrystalline cellulose, hydroxypropylmethyl cellulose, (a.2) at least one disintegrant, e.g. cross-linked polyvinylpyrrolidinone, e.g. Crospovidone®, (a.3) at least one glidant, e.g. colloidal silicon dioxide, (a.4) at least one lubricant, e.g. magnesium stearate and/or (a.5) basic coating.
41) The tablet according to any one of items 35 to 40, wherein darolutamide is present in an amount of from 400 mg to 900 mg darolutamide form I.
42) The tablet according to item 41, wherein the amount of darolutamide is from 500 mg to 800 mg darolutamide form I, such as 600 mg darolutamide form I.
43) The tablet according to any one of items 35 to 40, wherein the amount of darolutamide is from 180 mg to 540 mg darolutamide form II.
44) The tablet according to item 43, wherein the amount of darolutamide is from 250 mg to 500 mg, such as from 300 mg to 450 mg.

### EXAMPLES

The following non-limiting examples are illustrative for the disclosure and are not to be construed as to be in any way limiting for the scope of the invention.

### Example 1: Crystallization of Crystalline Form II of Darolutamide

Darolutamide (273 mg, e.g. prepared according to Example 56 of WO 2011/051540 A1) was dissolved in n-butanol (8.1 mL) upon heating to reflux temperature. Thereafter, the solution was cooled to a temperature of about 25° C. before n-heptane (40 mL) was added. The mixture was stirred for about 4 hours resulting in a homogenous suspension. The crystals were collected by filtration and dried under vacuum (30 mbar) and room temperature for 17.5 hours to obtain crystalline Form II of darolutamide (257 mg).

Powder X-Ray Diffraction

PXRD was performed with a PANalytical X'Pert PRO diffractometer equipped with a theta/theta coupled goniometer in transmission geometry, Cu-Kalpha_{1,2} radiation (wavelength 0.15419 nm) with a focusing mirror and a solid state PIXcel detector. Diffractograms were recorded at a tube voltage of 45 kV and a tube current of 40 mA, applying a stepsize of 0.013° 2-Theta with 40 s per step (255 channels) in the angular range of 2° to 40° 2-Theta at ambient conditions. A typical precision of the 2-Theta values is in the range of ±0.2° 2-Theta, preferably of ±0.1° 2-Theta. Thus, the diffraction peak of Form II of darolutamide that appears for example at 16.4° 2-Theta can appear in the range of from 16.2 to 16.6° 2-Theta, preferably in the range of from 16.3 to 16.5° 2-Theta on most X-ray diffractometers under standard conditions.

A representative diffractogram of crystalline Form II of darolutamide is displayed in FIG. 1 herein. The corresponding reflection list is provided in Table 1 below.

TABLE 1
PXRD reflections of crystalline Form II of
darolutamide in the range of from 2 to 30° 2-Theta;
A typical precision of the 2-Theta values is in the range
of ± 0.2° 2-Theta, preferably of ± 0.1° 2-Theta.

| Reflection position | Reflection position |
|---|---|
| [° 2-Theta] | [° 2-Theta] |
| 5.9 | 18.2 |
| 7.5 | 19.1 |
| 9.1 | 20.3 |
| 11.8 | 22.0 |
| 13.7 | 22.6 |
| 15.0 | 23.5 |
| 16.4 | 24.6 |
| 17.1 | 25.5 |
| 17.8 | 27.8 |

Fourier Transform Infrared Spectroscopy

The FTIR spectrum was recorded (obtained) on a MKII Golden Gate™ Single Reflection Diamond ATR cell with a Bruker Tensor 27 FTIR spectrometer with 4 cm⁻¹ resolution at a temperature in the range of from 20 to 30° C. To record a spectrum a spatula tip of the sample was applied to the surface of the diamond in powder form. Then the sample was pressed onto the diamond with a sapphire anvil and the spectrum was recorded. A spectrum of the clean diamond was used as background spectrum. A typical precision of the wavenumber values is in the range of ±2 cm 1. Thus, the infrared peak of crystalline Form II of darolutamide that appears for example at 2233 cm⁻¹ can appear between 2231 and 2235 cm⁻¹ on most infrared spectrometers under standard conditions.

A representative FTIR spectrum of crystalline Form II of darolutamide according to the present invention is displayed in FIG. 2 and the corresponding peak list is provided in Table 2 below

TABLE 2
FTIR peak list of crystalline Form II of darolutamide
according to the present invention; a typical
precision of the wavenumbers is in the range of ± 2 cm⁻¹.

| Wavenumber | Wavenumber | Wavenumber |
|---|---|---|
| [cm⁻¹] | [cm⁻¹] | [cm⁻¹] |
| 3406 | 1499 | 1047 |
| 3252 | 1426 | 967 |
| 3165 | 1392 | 937 |
| 2979 | 1334 | 891 |
| 2948 | 1256 | 821 |
| 2233 | 1230 | 760 |
| 1647 | 1200 | 694 |
| 1601 | 1132 | 636 |
| 1539 | 1081 | 995 |

Differential Scanning Calorimetry

DSC was performed on a Mettler Polymer DSC R instrument. The sample (3.72 mg) was heated in a 40 microliter aluminium pan with a pierced aluminium lid from 25 to 220° C. at a rate of 10 K/min. Nitrogen (purge rate 50 mL/min) was used as purge gas.

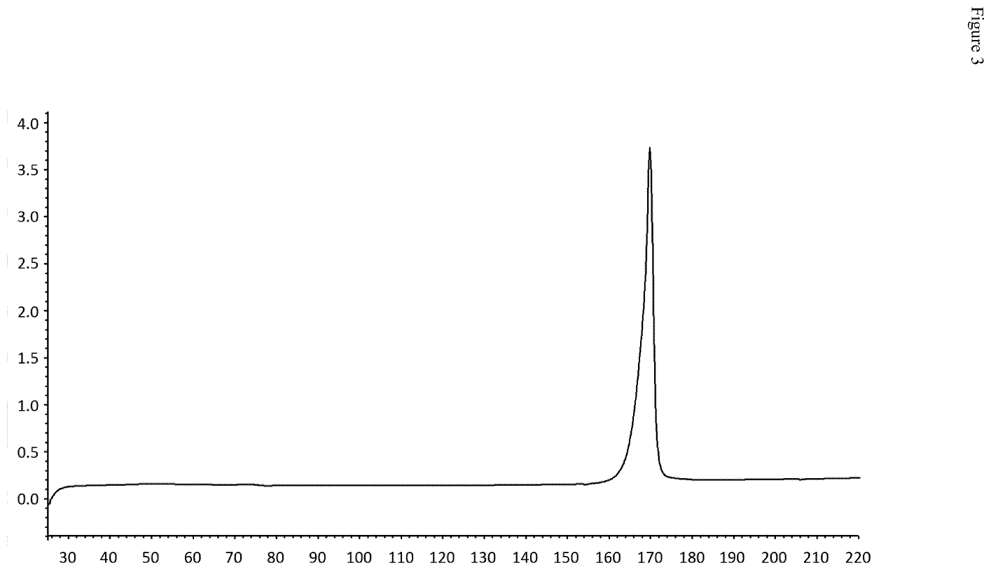

The DSC curve of Form II of darolutamide shows a single endothermic peak with an onset temperature of about 167° C., a peak temperature of about 169° C. and a heat of fusion of about 74 J/g (see also FIG. 3 herein). This peak is due to the melting of Form II.

Thermogravimetric Analysis

TGA was performed on a Mettler TGA/DSC 1 instrument. The sample (9.46 mg) was heated in a 100 microliter aluminum pan closed with an aluminum lid. The lid was automatically pierced at the beginning of the measurement. The sample was heated from 25 to 220° C. at a rate of 10 K/min. Nitrogen (purge rate 50 mL/min) was used as purge gas.

Form II shows a mass loss of only about 0.7 weight % until it melts at about 170° C. (see FIG. 4 herein). Hence, it can be concluded that neither water nor organic solvent is part of the crystal structure but the mass loss is caused by the release of loosely bound residual solvent and/or water.

### Example 2: Crystallization of Crystalline Form II of Darolutamide

Darolutamide (50 mg, e.g. prepared according to Example 56 of WO 2011/051540 A1) was dissolved in a solvent (Table 3) upon heating to reflux temperature. The solution was optionally filtered to eliminate any non-dissolved particles. Thereafter, the clear solution was cooled to a temperature of about 25° C. before an antisolvent (Table 3) was added. The mixture was stirred until a homogenous suspension occurred (Table 3). The crystals were collected by filtration and optionally dried under vacuum (30 mbar) and room temperature for 17.5 hours to obtain crystalline Form II of darolutamide.

TABLE 3
Amounts of used solvents and antisolvents of Example 2
and the stirring time after addition of the antisolvent.

| | | | | | stirring |
|---|---|---|---|---|---|
| | | | | | time after |
| | | | | | antisolvent |
| | | amount | | amount | addition |
| Example | solvent | [mL ] | antisolvent | [mL] | [h] |
| a | methanol | 1 | H₂O | 4 | 1 |
| b | 2-propanol | 2.5 | H₂O | 10 | 1 |
| c | acetone | 3 | H₂O | 40 | 1 |
| d | acetic acid | 0.5 | H₂O | 2 | 18 |
| e | 1,4-dioxane | 1 | H₂O | 40 | 1 |
| f | DMF | 0.25 | H₂O | 1 | 1 |
| g | tert-amylalcohol | 2.5 | n-heptane | 9 | 1 |
| h | methyl acetate | 3 | n-heptane | 9 | 1 |

### Example 3: Comparison of Solubility of Form I and Form II

a) Solubility in Aqueous Hydrochloric Acid (0.1 N)

Form I

102.39 mg of darolutamide form I were slurried in 20 mL aqueous hydrochloric acid (0.1 N) at 37° C.

Form II

99.41 mg of darolutamide form II were slurried in 20 mL aqueous hydrochloric acid (0.1 N) at 37° C.

b) Solubility in Phosphate Buffer (pH 7.5)

Form I

105.02 mg of darolutamide form I were slurried in 20 mL aqueous hydrochloric acid (0.1 N) at 37° C.

Form II

103.41 mg of darolutamide form II were slurried in 20 mL aqueous hydrochloric acid (0.1 N) at 37° C.

Samples were taken after 5 min, 15 min and 60 min. The suspensions were filtered and subjected undiluted to HPLC. The absolute solubility was calculated by creating a linear calibration curve (see below). Results are reported in Table 4 and in FIGS. 10 and 11.

TABLE 4
Concentrations in solution after 5, 15 and 60 min of slurry
in hydrochloric acid and phosphate buffer.

| | aqueous hydrochloric | | phosphate | |
|---|---|---|---|---|
| | acid (0.1N) | | buffer (pH 7.5) | |
| slurry | Form I | | Form I | |
| time | solubility | Form II | solubility | Form II |
| [min] | [μg/mL] | [μg/mL] | [μg/mL] | [μg/mL] |
| 5 | 43 | 113 | 43 | 89 |
| 15 | 41 | 121 | 40 | 97 |
| 60 | 38 | 117 | 42 | 93 |

c) Calibration Curve for Solubility Measurements

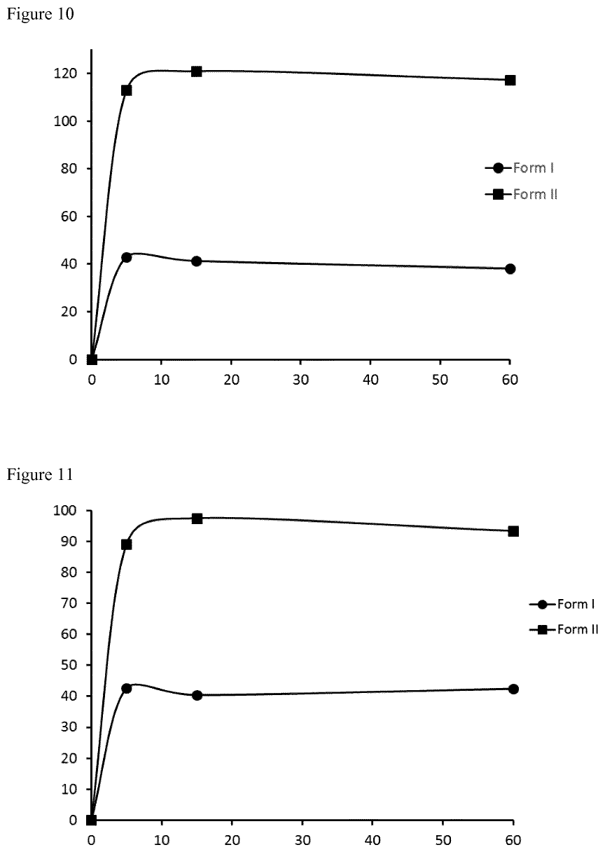

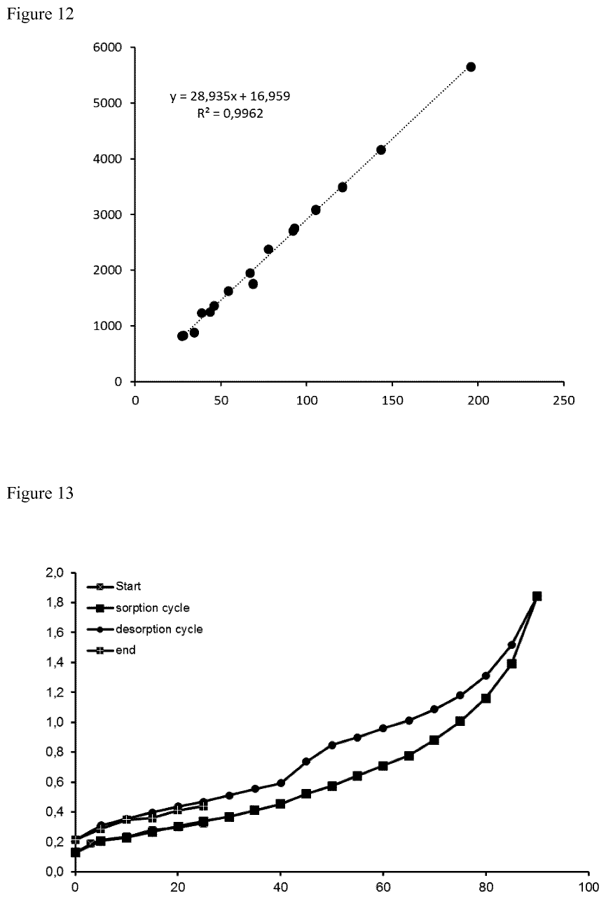

Solutions of different concentrations (Table 5) of darolutamide were prepared in acetonitrile/water (75% v/v) at 37° C. and subjected to HPLC measurement. The resulting areas were illustrated as a function of the concentrations and a linear dependency could be observed (FIG. 12).

TABLE 5
Table of concentrations of the standard
solutions and the peak areas.

| concentration | area | concentration | area |
|---|---|---|---|
| [μg/mL] | [a.u.] | [μg/mL] | [a.u.] |
| 27 | 823 | 69 | 1750 |
| 27 | 826 | 69 | 1765 |
| 28 | 833 | 78 | 2377 |
| 28 | 734 | 78 | 2381 |
| 34 | 886 | 92 | 2714 |
| 34 | 895 | 92 | 2709 |
| 39 | 1239 | 93 | 2754 |
| 39 | 1232 | 93 | 2764 |
| 43 | 1257 | 105 | 3075 |
| 43 | 1255 | 105 | 3100 |
| 46 | 1361 | 121 | 3503 |
| 46 | 1368 | 121 | 3492 |
| 54 | 1638 | 143 | 4167 |
| 54 | 1629 | 143 | 4163 |
| 67 | 1955 | 196 | 5659 |
| 67 | 1951 | 196 | 5652 |

The linear trendline exhibits the function y=28.935×+16.959 (R²=0.9962), wherein y represents the peak areas and x the concentration of the solutions. Hence, the equilibrium solubility is calculated:

$$x = \frac{y - 16.959}{28.935}$$

HPLC parameters:

Column: YMC-Pack Pro C18 RS; 150 mm*4.6 mm; 3 μm

Eluent A: 3.88 gsulfamic acid ad 1000 gH₂O

Eluent B: 3.88 gsulfamic acid ad 300 gH₂O+587 gacetonitrile

Injection volume: 5 μL at 40° C.

Flow: 0.8 mL/min

Oven temperature: 40° C.

Wavelength: 210 nm

Gradient program:

| t [min] | % eluent A | % eluent B |
|---|---|---|
| 0 | 90 | 10 |
| 10 | 0 | 100 |
| 15 | 0 | 100 |
| 16 | 90 | 10 |

Retention time: 9.8 min

### Example 4: Gravimetric Moisture Sorption (GMS)

Moisture sorption isotherms were recorded with an SPSx-1p moisture sorption analyzer (ProUmid, Ulm. The measurement cycle was started at a relative humidity (r.h.) of 25%. Relative humidity was then decreased to 5% r.h. in 5% steps, followed by a further decrease to 3% r.h. and to 0% r.h. Afterwards r.h. was increased from 0% to approximately 90% r.h. in a sorption cycle and decreased to 0% in a desorption cycle in 5% steps. Finally r.h. was increased to 25% r.h. in 5% steps.

The time per step was set to a minimum of 2 hours and a maximum of 6 hours. If an equilibrium condition with a constant mass of 0.01% within 1 hour was reached before the maximum time for all examined samples the sequential humidity step was applied before the maximum time of 6 hours. If no equilibrium was achieved the consecutive humidity step was applied after the maximum time of 6 hours. The temperature was 25±0.1° C.

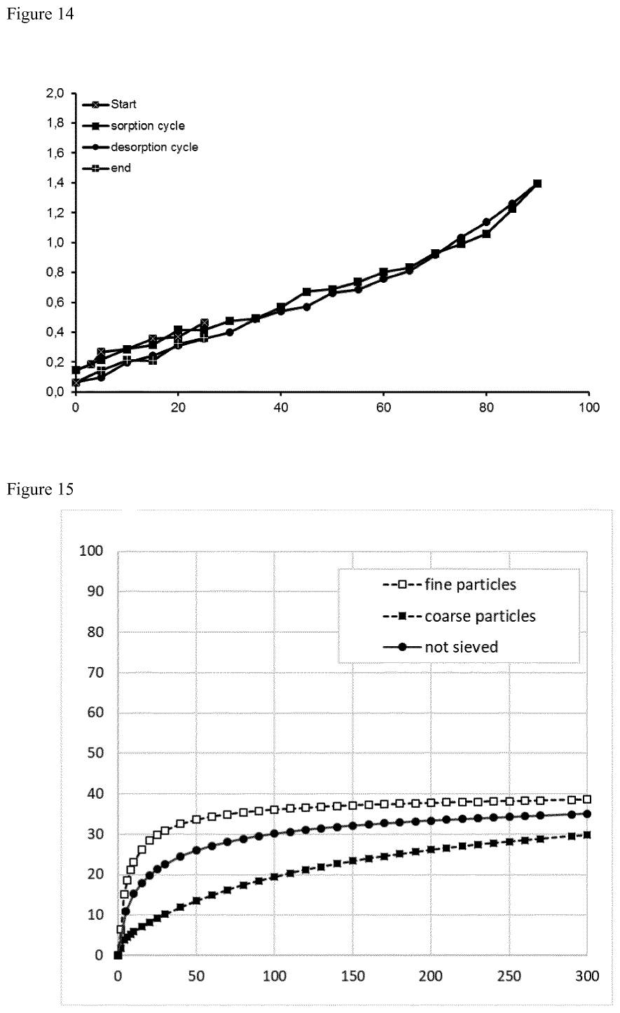

Neither form I nor form II underwent any phase change during the GMS experiments (see FIGS. 13 and 14), but form II resulted to be less hygroscopic than form I.

| | water uptake from 0% to 90% RH |
|---|---|
| form I | +1.7% |
| form II | +1.3% |

### Example 5: Influence of Darolutamide Form I Particle Size on the Tablet Dissolution Profile

a) Fractional Sieving of Darolutamide Form I

Darolutamide form I was fractionated according to particle sizes via sieving, using a mechanical sieve shaker (Retsch AS300 control) with the following set up: The nested column of four different meshed sieves, was stacked on a mechanical shaker. Following mesh sizes were used (in top to bottom order): 800 μm, 500 μm, 150 μm, 125 μm. The API was then put on the top sieve (800 μm) and sieving was performed at different amplitudes until constant weightings of each sieve was achieved (<2% difference).

b) Tableting:

An un-sieved fraction and two sieved fractions, coarse particles (800 μm>x>500 μm) and fine particles (250 μm>x>125 μm), were pressed separately into tablets (300 mg) according to the below listed formula. All additional components of the tablet mixtures were first individually sieved through an 800 μm mashed sieve and then thoroughly mixed together using a TURBULA® T2F three dimensional shaker-mixer (WAB) for 5 min (level 34). Tablets were then pressed on a single punch tablet press Flexitab S (Röltgen Marking System) with a 9×19 mm die (lower punch height: 9 mm) using 100 bar compaction pressure (hold time: 0 sec; return time: 3 sec).

| | [g] | [%] |
|---|---|---|
| Darolutamide Form I | 0.307 | 30% |
| Lactose monohydrate | 0.410 | 41% |
| Cellulose microcrystalline (MCC) | 0.220 | 22% |
| Croscarmellose sodium | 0.030 | 3% |
| SDS (Sodium dodecylsulfate) | 0.020 | 2% |
| Aerosil (silicium dioxide) | 0.005 | 0.5% |
| Magnesium stearate | 0.0075 | 0.75% |
| SUM: | 1.000 | |

c) Dissolution Analysis:

Dissolution was performed on a paddle apparatus (Agilent) according to USP II. Following conditions were used: FeSSIF, pH 5.0, 900 mL, 37° C., 75 rpm, detection at 289 nm wavelength.

We observed here that tablets prepared from fine particles of daroluatmide form I showed superior dissolution kinetics over tablets from unsieved material and even more so over tablets prepared from coarse particles. FIG. 15 shows that tablets prepared from fine particles dissolved much more quickly under fed conditions (FeSSIF) than tablets prepared from larger particles, which was surprising in view of Massard et al., European Urology 69 (2016), 834-840, as FIG. 2 on page 837 of Massard showed at best marginal differences in pharmacokinetic studies with formulations prepared from different particle sizes of the drug substance.

### Example 6: Comparison of 600 mg Tablets with Un-Sieved and Fine Particles of Darolutamide Form I

a) Fractional sieving of darolutamide form I:

Daroluatmide form I particles were separated via sieving and the fraction <150 μm (fine particles) were collected.

b) Tableting

The fine particles and un-sieved API were pressed separately into tablets (600 mg, corresponding to 70% or 80% drug load, respectively) according to below listed formula. All additional components of the tablet mixtures were first individually sieved through an 800 μm mashed sieve and then thoroughly mixed together using a TURBULA® T2F three dimensional shaker-mixer (WAB) for 5 min (level 34). Tablets were then pressed on a single punch tablet press Flexitab S (Röltgen Marking System) with a 9×19 mm die (lower punch height: 9 mm) using 100 bar compaction pressure (hold time: 0 sec; return time: 3 sec).

| | 70% | | 80% | |
|---|---|---|---|---|
| | [g] | [%] | [g] | [%] |
| Darolutamide form I | 0.600 | 70 | 0.600 | 80 |
| Lactose monohydrate | 0.148 | 17 | 0.068 | 9 |
| Cellulose microcrystalline (MCC) | 0.050 | 6 | 0.020 | 3 |
| Croscarmellose sodium | 0.030 | 3 | 0.030 | 4 |
| SDS (Sodium dodecylsulfate) | 0.020 | 2 | 0.020 | 3 |
| Aerosil (silicium dioxide) | 0.005 | 0.6 | 0.005 | 0.7 |
| Magnesium stearate | 0.0075 | 0.9 | 0.0075 | 1 |
| SUM [g]: | 0.860 | | 0.750 | |

c) Dissolution Analysis:

Dissolution was performed on a paddle apparatus (Agilent) according to USP II. Following conditions were used: Phosphate buffer pH 7.0+2.0 wt % SDS, 900 mL, 37° C., 75 rpm, detection at 252 nm wavelength.

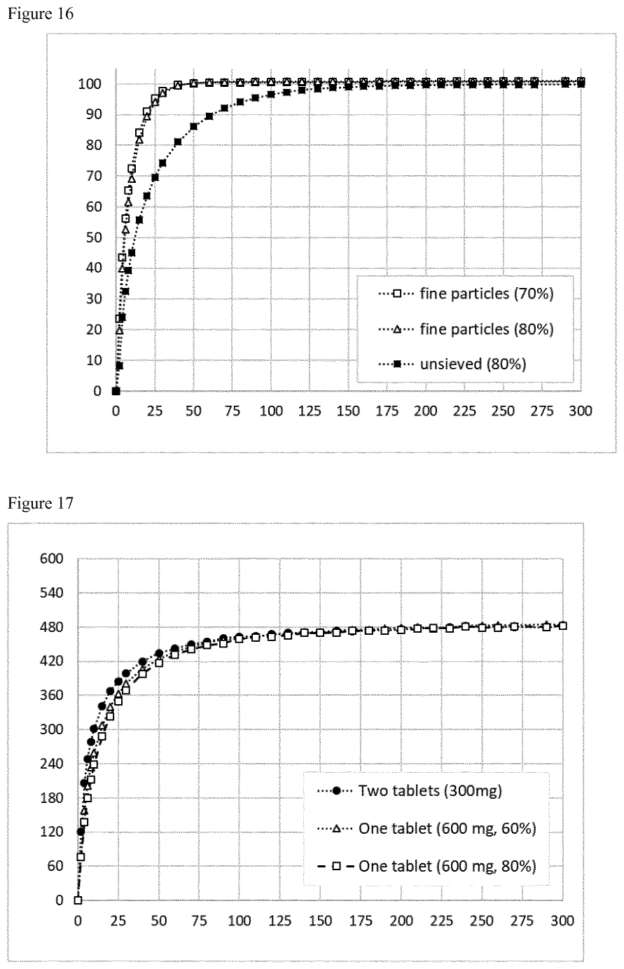

As shown in FIG. 16, the use of fine particles of darolutamide allowed to increase the drug load in the tablet to more than 60% w/w while still displaying a very fast release profile for the drug substance.

### Example 7: Comparison of the Dissolution Profiles of Two 300 mg Tablets with One 600 mg Tablet Containing Darolutamide Form II

a) Tableting of 300 and 600 mg Tablets

Darolutamide form II particles were separated via sieving and the fraction <150 μm (fine particles) were collected and pressed into tablets (300 mg and 600 mg, respectively with different drug load) according to the below listed formula. All additional components of the tablet mixtures were first individually sieved through an 800 μm mashed sieve and then thoroughly mixed together using a TURBULA® T2F three dimensional shaker-mixer (WAB) for 5 min (level 34). Tablets were then pressed on a single punch tablet press Flexitab S (Röltgen Marking System) with a 9×19 mm die (lower punch height: 9 mm) using 100 bar compaction pressure (hold time: 0 sec; return time: 3 sec).

| | 300 mg | | 600 mg (60%) | | 600 mg (80%) | |
|---|---|---|---|---|---|---|
| | [g] | [%] | [g] | [%] | [g] | [%] |
| Darolutamide form II | 0.300 | 30 | 0.600 | 60 | 0.600 | 80 |
| Lactose monohydrate | 0.403 | 40 | 0.220 | 22 | 0.068 | 9 |
| Cellulose microcrystalline | 0.220 | 22 | 0.118 | 12 | 0.020 | 3 |
| (MCC) | | | | | | |
| Croscarmellose sodium | 0.030 | 3 | 0.030 | 3 | 0.030 | 4 |
| SDS (Sodium dodecylsulfate) | 0.020 | 2 | 0.020 | 2 | 0.020 | 3 |
| Aerosil (silicium dioxide) | 0.005 | 0.5 | 0.005 | 0.5 | 0.005 | 0.7 |
| Magnesium stearate | 0.0075 | 0.75 | 0.0075 | 0.75 | 0.0075 | 1 |
| SUM [g]: | 1.000 | | 1.000 | | 0.750 | |

b) Dissolution:

Dissolution was performed on a paddle apparatus (Agilent) according to USP II. Following conditions were used: FeSSIF, pH 5.0, 900 mL, 37° C., 75 rpm, detection at 289 nm wavelength.

As shown in FIG. 17, darolutamide form II is especially suitable for the formulation of tablets with high drug loads. We could show that 600 mg tablets with drug loads up to 80% display essentially the same fast dissolution profile as two 300 mg tablets having a much lower drug content of only 30% w/w.

1. A crystalline form of N-{(2S)-1-[3-(3-chloro-4-cyanophenyl)-1H-pyrazol-1-yl]-propan-2-yl}-5-[(1RS)-1-hydroxyethyl]-1H-pyrazole-3-carboxamide (Form II) characterized by having a powder X-ray diffractogram comprising reflections at 2-Theta angles of (5.9±0.2)°, (9.1±0.2)° and (16.4±0.2)°, when measured at a temperature in the range of from 20 to 30° C. with Cu-Kalpha_{1,2} radiation having a wavelength of 0.15419 nm.

2. The crystalline form of claim 1 characterized by having a powder X-ray diffractogram comprising additional reflections at 2-Theta angles of (7.5±0.2)° and (13.7±0.2)°, when measured at a temperature in the range of from 20 to 30° C. with Cu-Kalpha_{1,2} radiation having a wavelength of 0.15419 nm.

3. The crystalline form of claim 1 characterized by having a powder X-ray diffractogram comprising no reflections at 2-Theta angles of (8.5±0.2)° and (10.4±0.2)°, when measured at a temperature in the range of from 20 to 30° C. with Cu-Kalpha_{1,2} radiation having a wavelength of 0.15419 nm.

4. A process for the preparation of the crystalline form as defined in claim 1 comprising:
(i) dissolving darolutamide in a solvent selected from the group consisting of methanol, 2-propanol, n-butanol, tert-amylalcohol, acetone, methyl acetate, 1,4-dioxane, dimethylformamide and acetic acid or any mixture thereof;
(ii) adding an antisolvent selected from an alkane or water to the solution obtained in step (i) or vice versa, characterized in that the temperature of the mixture is maintained in the range of from 20 to 30° C. during the addition.

5. Use of the crystalline form as defined in claim 1 for the preparation of a pharmaceutical composition.

6. A pharmaceutical composition comprising the crystalline form as defined in claim 1, and at least one pharmaceutically acceptable excipient.

7. The pharmaceutical composition of claim 6, wherein the oral solid dosage form is a tablet.

8. The tablet of claim 7, wherein the amount of darolutamide,
calculated as the percentage of the content of darolutamide in weight based on the total weight of the tablet, is at least 50 percent.

9. The tablet of claim 8, wherein the darolutamide content is from 55 percent to 90 percent based on the total weight of the tablet.

10. The pharmaceutical composition according to claim 6 for use in the treatment and/or prophylaxis of prostate cancer.

11. The pharmaceutical composition of claim 10, wherein from 180 mg to 540 mg darolutamide form II are to be administered twice daily such that the total daily dose of darolutamide form II is from 360 mg to 1080 mg.
